SEQUENCE LISTING (2 sequences)

SEQ ID NO: 1 - protein, 121 aa, Homo sapiens
MARRAGGARMFGSLLLFALLAAGVAPLSWDLPEPRSRASKIRVHSRGNLWATGHFMGKKSLEPSSPSHWGQLPTPPLRDQRLQLSHDLLGILLLKKALGVSLSRPAPQIQYRRLLVQILQK

SEQ ID NO: 2 - protein, 148 aa, Homo sapiens
MRGRELPLVLLALVLCLAPRGRAVPLPAGGGTVLTKMYPRGNHWAVGHLMGKKSTGESSSVSERGSLKQQLREYIRWEEAARNLLGLIEAKENRNHQPPQPKALGNQQPSWDSEDSSNFKDVGSKGKVGRLSAPGSQREGRNPQLNQQ

(19) United States
(12) Patent Application Publication
(10) Pub. No.: US 2017/0224778 A1
(43) Pub. Date: Aug. 10, 2017

(54) COMPOSITIONS AND METHODS THAT TARGET REGULATION OF BREATHING

(71) Applicants: The Board of Trustees of the Leland Stanford Junior University, Stanford, CA (US); The Regents of the University of California, Oakland, CA (US)

(72) Inventors: Peng Li, Union City, CA (US); Mark Alan Krasnow, Stanford, CA (US); Kevin Yackle, San Francisco, CA (US); Wiktor A. Janczewski, Simi Valley, CA (US); Jack Feldman, Los Angeles, CA (US)

(21) Appl. No.: 15/424,668

(22) Filed: Feb. 3, 2017

Related U.S. Application Data
(60) Provisional application No. 62/292,156, filed on Feb. 5, 2016.

(51) Int. Cl. A61K 38/22; A61K 38/10

(52) U.S. Cl. CPC A61K 38/22; A61K 38/105

(57) ABSTRACT

Activation of neurons in the preBötC with neuromedin B or gastrin releasing peptide increases frequency of sighs.

### CROSS REFERENCE

This application claims benefit U.S. Provisional Patent Application No. 62/292,156, filed Feb. 5, 2016, which application is incorporated herein by reference in its entirety.

### BACKGROUND OF THE INVENTION

A sigh is a long deep breath often associated with sadness, yearning, exhaustion or relief. Sighs also occur spontaneously, from several per hour in humans to dozens per hour in rodents. Their recurrence during normal breathing enhances gas exchange and may preserve lung integrity by reinflating collapsed alveoli. Sighing increases in response to emotional and physiological stresses, including hypoxia and hypercapnia, and in anxiety disorders and other psychiatric conditions where it can become debilitating.

The kernel of the breathing rhythm generator is the preBötC, a cluster of several thousand neurons in ventrolateral medulla. preBötC is required for inspiration and generates respiratory rhythms in explanted brain slices. Each rhythmic burst activates premotoneurons and motoneurons that contract the diaphragm and other inspiratory muscles, generating a normal (“eupneic”) breath. Occasionally, a second preBötC burst immediately follows the first, and this “double burst” leads to the augmented inspiration of a sigh, typically about twice the volume of a normal breath. Thus, the command for normal breaths and sighs both appear to emanate from preBötC. A variety of neuromodulators and neuropeptides, including frog bombesin, can influence sighing in rodents. However, the endogenous sigh control pathways have not been identified.

Breathing is an essential component of life, and must be maintained from the first to the last breath. Disturbances in the neuronal control of breathing have devastating consequences and may ultimately be fatal. Various neurological conditions are associated with severe breathing disturbances. Thus, understanding how breathing is generated within the nervous system and how the CNS controls ventilatory functions is of great clinical interest.

### PUBLICATIONS

Gray et al. Nat. Neurosci. 4, 927-930 (2001). Tan et al. Nat. Neuroscience. 5, 538-540 (2008). Feldman et al. Annu. Rev. Physiol. 75, 423-452 (2013). Kam et al. J. Neurosci. 33, 9235-9245 (2013). Lieske et al. Nat. Neurosci. 3, 600-607 (2000). Ruangkittisakul et al. J. Neurosci. 28, 2447-2458 (2008).
Jensen et al., International Union of Pharmacology. LXVIII. Pharmacol Rev. 60, 1-42 (2008). Ohki-Hamazaki et al. J. Neurosci. 19, 948-954 (1999).

### SUMMARY OF THE INVENTION

Compositions and methods are provided for modulating of sigh breathing in a mammal, through inhibiting or activating the neural receptors that control sighs. It is shown herein that activation of neuromedin B (NMB) or gastrin releasing peptide (GRP) receptors in preBötC neurons controls sighs in breathing. Activation of NMBR or GRPR increases the frequency of sighs in breathing, while inhibitors of the receptor can diminish the frequency of sighs.

In one embodiment of the invention, a method is provided for modulating sigh breathing, the method comprising contacting cells of the preBötC in a subject with an effective of an agonist or antagonist of NMBR or GRPR. An effective dose of an agonist is sufficient to increase the frequency of sighs in the subject, relative to the level of ventilation in the absence of the agonist. For example, sigh frequency may be increased by about 5%, by about 10%, by about 15%, by about 20%, by about 25%, by about 30%, by about 35%, by about 40%, by about 45%, by about 50%, by about 55%, by about 65%, by about 75%, by about 85%, by about 95%, by about 100%, or more, e.g. 2-fold, 3-fold, etc. An effective dose of an antagonist is sufficient to counteract dysregulated signaling that results in undesirable hyperventilation.

In one embodiment, methods are providing for screening candidate agents for activity in modulation of sigh breathing. In some embodiments the candidate agent is screened for activity against NMBR or GRPR. Candidate agents can be initially screened by contacting with the receptor, including cells expressing the receptor, and determining changes in binding to the receptor, release of cyclic AMP, induction of motor neuron output, and the like. Agents that modulate the receptor may be further screened for activity in regulating the ventilator response of an animal. Candidate antagonists include anti-sense RNAs and RNAi specific for NMB, NMBR, GRP, GRPR; or antibodies that specifically bind to NMB, NMBR, GRP, GRPR without activating the receptor; and the like.

In some embodiments the methods of the invention are utilized in regulating sigh responses in sleep disordered breathing, during forced ventilation, in conditions of hyper- or hypo-ventilation, and the like.

For the methods of the invention, administration of an effective dose of an agent that regulates breathing may be provided acutely, e.g. for a period of from about 10 minutes, from about 20 minutes, from about 30 minutes, from about one hour, from about 2 hours, from about 4 hours, from about 6 hours, from about 8 hours, from about 12 hours to about 24 hours, to about 18 hours, to about 16 hours, to about 14 hours, to about 12 hours, to about 10 hours, to about 8 hours, to about 6 hours, to about 4 hours, to about 1 hour. Alternatively, for certain conditions it may be desirable to provide an effective dose of an agent for extended periods of time, e.g. from about 1 day, about 2 days, about 3 days, about 4 days, about 5 days, about 1 week, about 2 weeks, about 3 weeks, about 4 weeks, or more.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1. NMB neuropeptide pathway neurons in breathing center. (a), P0 mouse brain section probed for Nmb mRNA (green) with DAPI counterstain (nuclei, blue). Bar, 1 mm. (b), Boxed region (a) showing specific expression in RTN/pFRG. Bar, 100 μm. (c), Whole mount P0 brainstem (ventral view) showing Nmb-GFP transgene expression (GFP, green) bilaterally in RTN/pFRG. Bar, 0.5 mm. (d),(e), 3D reconstruction (sagittal (d), coronal (e) projections) of CLARITY-cleared P14 Nmb-GFP brainstem. Note RTN/pFRG expression ventral, dorsal, and lateral to facial nucleus. Numbers, representative neurons. A, anterior; V, ventral; M, medial. Bar, 100 μm. (f), P0 Nmb-GFPexpressing neurons (green) in RTN/pFRG (dashed) co-express RTN marker PHOX2B (red). Bar, 50 μm. (g), P7 Nmb-GFP-expressing neurons (green) project to preBötC (dashed). SST (somatostatin), preBötC marker (white). *, isolated GFP-labeled neuron in facial nucleus. Bar, 100 μm. (h), Boxed region (g) with NMB co-stain (z-stack projection; optical sections, FIG. 7). Arrowhead, NMB puncta (red) in Nmb-GFP-expressing projection (green) abutting preBötC neuron (SST, white). Bars, 10 μm (1 μm, inset). (i), P7 ventral medulla section probed for Nmbr mRNA (purple) showing preBötC expression. Bar, 100 μm. (j), Tiled image (left) and tracing (right) of Nmb-GFP neuron as in (g) projecting to preBötC. Bar, 30 μm.

FIG. 2. NMB effect on breathing. (a-c), Breathing activity of anesthetized rat following bilateral NMB injection (100 nl, 3 μM) into preBötC. Note increased sighing (spikes in tidal volume (V_{T}), integrated diaphragm activity (∫Dia)) but little change in respiratory rate (frequency, f). Bar, 1 min. (b,c), Similar, stereotyped waveforms of spontaneous (b) and NMB-induced (c) sighs (from a; also FIG. 8a,c-f). Bar, 2 s. (d), Quantification of (a). Top: raster plots of sighs (tics) in five rats following NMB injection (grey); numbers, highest instantaneous sigh rate (red tics). Bottom: instantaneous sigh rate of bottom raster plot; numbers, average instantaneous sigh rate before and maximum (and fold increase) after injection. (e), Integrated hypoglossal nerve (∫XII; black) and preBötC neural activity (∫preBötC; grey) in preBötC slices containing indicated NMB concentrations. NMB increases doublets (*), a sigh signature in slices. Bar, 10 s. (f), Quantification of (e) (n=7; *, p<0.05). (g), Basal sigh rate in C57BL/6 wild-type (WT) and Nmbr−/− mice. n=4; bars, standard deviation of mean; *, p<0.001. (h), Effect on sighing in anesthetized rats of bilateral preBötC injection (grey) of NMBR antagonist BIM23042 (100 nl, 6 μM). Top: raster plots; numbers, longest intersigh intervals (s, seconds) following injection. Bottom: sliding average sigh rate (bin 4 min; slide 30 s); numbers, average rate before (left) and minimum binned rate after injection (right).

FIG. 3. GRP neuropeptide pathway expression and function in breathing. a-b, Sagittal ventral medulla sections of P7 mice probed for Grp (a) or Grpr (b) mRNA (purple). Bar, 200 μm. (c), Effect on sighing of bilateral preBötC injection of GRP (100 nl, 3 μM), as in FIG. 2d. (d), Effect of GRP on doublets (sighs) in preBötC slices, as in FIG. 2f. n=9; *, p<0.05. (e), Basal sigh rate in C57BL/6 wild-type (WT) and Grpr−/− mice, as in FIG. 2g. (f), Effect on sighing of bilateral preBötC injection of GRPR antagonist RC3095 (100 nl, 6 μM), as in FIG. 2h.

FIG. 4. Interactions between NMB and GRP pathways in sighing. (a-d), RTN/pFRG section of P7 Nmb-GFP mouse immunostained for GFP (green, arrowheads) and probed for Grp mRNA (red, arrows). Note no expression overlap. Bar, 30 μm. (e-h), preBötC section of P28 mouse probed for Nmbr mRNA (green, arrowheads) and Grpr mRNA (red, arrows). Note partial expression overlap. Bar, 30 μm. (i), Effect on sighing of bilateral preBötC injection of both NMB (100 nl, 3 μM) and GRP (100 nl, 3 μM) as in FIG. 2d. (j), Effect on sighing of bilateral preBötC injection (100 nl, 6 μM) of both NMBR and GRPR antagonists (BIM23042, RC3095) as in FIG. 2h.

FIG. 5. Effect on sighing of ablating preBötC NMBR-expressing and GRPRexpressing neurons. (a,b), Basal (a) and hypoxia-induced (b) sigh rates before (control) and 3 or 5 days after preBötC injections of bombesin-saporin (200 nl, 6.2 ng; BBN-SAP ablation) to ablate NMBR and GRPR expressing neurons, or 5 days after saporin alone (200 nl, 6.2 ng; Blank-SAP). (c), Model of peptidergic sigh control circuit. NMB- and GRPexpressing neurons in RTN/pFRG (and perhaps GRP-expressing neurons in NTS and PBN) receive physiological and perhaps emotional input from other brain regions, stimulating neuropeptide secretion. This activates receptor-expressing preBötC neurons expressing their receptors, which transform the normal preBötC rhythm to sighs. (Because neuropeptides induce sighs separated by normal breaths (FIG. 2A), there must be some refractory mechanism in or downstream of receptor-expressing neurons that temporarily prevents a second sigh.)

FIG. 6. Expression of neuromedin b (Nmb) in rodent brain. (a, b), Sagittal sections of P7 mouse (a) and P7 rat (b) brain showing RTN/pFRG region probed for Nmb mRNA expression (purple) by in situ hybridization as in FIG. 1. Bars, 100 μm. (c, d), Nmb expression as in a showing regions outside ventrolateral medulla. Nmb is expressed in mouse olfactory bulb (c) and hippocampus (d). Bars, 200 μm (c) and 100 μm (d). (e-h), Section through RTN/pFRG brain region of P0 transgenic Nmb-GFP mouse immunostained for GFP (green) and probed for Nmb mRNA (red) by in situ hybridization. Blue, DAPI nuclear stain. Nmb-GFP and Nmb mRNA are largely coexpressed in same cells. Bar, 100 μm.

FIG. 7. Serial confocal preBötC sections showing Nmb-GFP projections contain puncta of NMB. (a-d), Serial confocal optical sections (0.6 [symbol] m apart) through preBötC brain region of Nmb-GFP mouse immunostained for GFP (green), NMB (red), preBötC marker SST (white), and DAPI (blue) as in FIG. 2h. Note the GFP-positive projection with a puncta of NMB (yellow, open arrows in (b), (c)) directly abutting an SST positive neuron (asterisk). Most NMB puncta (open arrowheads) were detected within GFP-positive projections as expected, and only a small fraction of NMB puncta (closed arrowhead) were detected outside them; NMB outside Nmb-GFP projections could be secreted protein or the rare Nmb-expressing cells that do not coexpress the Nmb-GFP transgene (see Extended Data FIG. 1e-h). Bar, 20 μm.

FIG. 8. Sighing after surgery and bilateral injection of saline into preBötC. (a), Example of a sigh in a breathing activity trace of a urethane-anesthetized rat after surgery as in FIG. 2a-c. V_{T}, tidal volume; ∫Dia, integrated diaphragm activity; Dia, raw diaphragm activity trace. (b), Sigh rate before (control) and after (saline) bilateral saline injection into preBötC. There is no effect of saline injection (n=5, p=0.83). (c-f), Breathing activity trace as in a (but also showing airflow). Note stereotyped waveform of sighs (d-f). Bars, 1. min (c), 1 second (d-f).

FIG. 9. Effects on sighing in individual rats following bilateral injection into preBötC of NMB, GRP and both NMB/GRP. (a-e), Raster plot of sighs (upper) and instantaneous sigh rates (lower) before and after NMB injection for the five experiments (a-e) shown in FIG. 2d. (f-j), Raster plot of sighs (upper) and instantaneous sigh rates (lower) before and after GRP injection for the five experiments (f-j) shown in FIG. 3c. k-o, Raster plot of sighs (upper) and instantaneous sigh rates (lower) before and after NMB/GRP injection for the five experiments (k-o) shown in FIG. 4i. Grey, injection period; arrowhead in raster plots, maximum instantaneous sigh rate; numbers, basal (left) and maximal instantaneous sigh rate (right) and fold induction (in parentheses) after neuropeptide injection.

FIG. 10. Effect of NMB on rhythmic activity of preBötC slice. Li et al, p. 3 4 (a), Neuronal activity trace (∫XII, black; ∫preBötC population activity, grey) of preBötC slice containing 30 nM NMB, as in FIG. 2e. Note the extreme effect of NMB in which every burst (“breath”) in the trace is a doublet (“sigh”, *). Bar, 5 s. (b,c), NMB increases the doublet rate by increasing the fraction of total events that are doublets (b) and decreasing the interval following a doublet (c). *, p<0.05, n=7. d,e, GRP also increases the doublet rate by increasing the fraction of total events that are doublets (d) and decreasing the interval following a doublet (e). *, p<0.05, n=9. Note that post-doublet intervals are significantly longer than post-burst intervals under all conditions, consistent with longer post-sigh apneas in vivo.

FIG. 11. Effects on sighing in individual rats following bilateral injection of BIM23042, RC3095 and BIM23042/RC3095 into preBötC. (a-d), Raster plot of sighs (upper) and binned sigh rates (lower; bin size 4 min; slide 30 s) before and after injection of the NMBR antagonist BIM23042 for the four experiments shown in FIG. 2h. (e-h), Raster plot of sighs (upper) and binned sigh rates (lower; bin size 4 min; slide 30 s) before and after injection of the GRPR antagonist RC3095 for the four experiments shown in FIG. 3f. (i-n), Raster plot of sighs (upper) and binned sigh rates (lower; bin size 4 min; slide 30 s) before and after BIM23042 and RC3095 injection for the six experiments shown in FIG. 4j. Grey, injection period; numbers, longest intersigh intervals (s, seconds) following injection.

FIG. 12. Specificity of antagonists BIM23042 and RC3095 in preBötC slice. (a), BIM 23042 (100 nM) blocks the effect of NMB (10 nM), but not GRP (3 nM) in preBötC slices. *, p<0.05, n=7. (b), RC3095 (100 nM) shows the opposite specificity, blocking the effect of GRP (3 nM), but not NMB (10 nM). *, p<0.05, n=9.

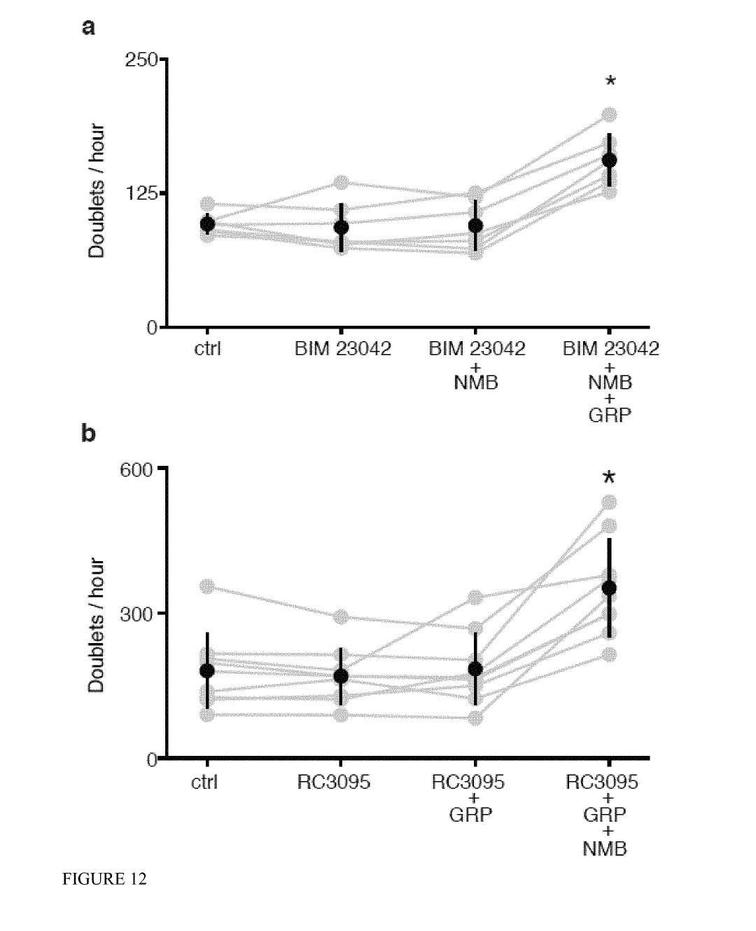

FIG. 13. Expression of Grp in rodent brain. (a,b), In situ hybridization of mouse brain slices as in FIG. 3a showing expression of Grp (purple) in parabrachial nucleus (PBN) (a) and nucleus tractus solitarius (NTS) (b). Bar, 200 μm. (c-e), In situ hybridization of rat brain slices showing expression of Grp in PBN (c), NTS (d), RTN/pFRG (e). Bar, 200 μm. (f), Tiled image showing GRP-positive projection (red) from RTN/pFRG region to preBötC region containing SST-positive neuron (green). Bar, 20 μm. (g-i), Serial confocal optical sections (0.8 μm apart) through mouse preBötC stained for GRP (red) and SST (green) focusing on short segment of GRP-positive projection where a GRP puncta (red) directly abuts (arrowhead) an SSTpositive neuron. Bar, 10 μm.

FIG. 14. Effect of bombesin injection on sighing following bombesin-saporin (BBN-SAP)-induced ablation of NMBR and GRPR-expressing preBötC neurons. (a, b), 10 min plethysmography traces of a control rat (a) and a day 5 BBN-SAP injected rat (b) during eupneic breathing (left). Indicated parts (10 secs) of traces are expanded at Li et al, p. 3 6 right. Note presence of sighs with stereotyped waveform in control rat, and no sighs detectable in BBN-SAP injected rat. (c), Sigh rate before (Control) and after 10 μg bombesin injection (BBN) into the cisterna magna of rats prior to BBN-SAP injection (WT) and at day 4 and day 6 after BBN-SAP injection (BBN-SAP) into the preBötC to ablate NMBR and GRPR-expressing neurons as in FIG. 5a, b. Values shown are mean±S.D. (WT, n=10; BBN-SAP, n=7 for day 4 and n=5 for day 6), *, p<0.05; n.s., not significant.

### DETAILED DESCRIPTION OF THE EMBODIMENTS

The following detailed description refers to the accompanying drawings which show, by way of illustration, specific aspects and embodiments in which the present invention may be practiced. The various embodiments are not necessarily mutually exclusive, as aspects of one embodiment can be combined with aspects of another embodiment. These embodiments are described in sufficient detail to enable those skilled in the art to practice the invention. Other embodiments may be utilized and structural, logical, and electrical changes may be made without departing from the scope of the present invention.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Methods recited herein may be carried out in any order of the recited events which is logically possible, as well as the recited order of events.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications mentioned herein are incorporated herein by reference to disclose and describe the methods and/or materials in connection with which the publications are cited.

It must be noted that as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

A sigh, as used herein, refers to a deep augmented breath with distinct neurobiological, physiological, and psychological properties that distinguish it from a normal eupneic breath. Sighs are biphasic breaths with two distinct components: the first burst resembles a normal eupneic breath, and the second burst is an augmented inspiratory peak initiated on the top of the first burst. Sighs are typically followed by a respiratory pause, which is referred to as “postsigh apnea.” Sighs have important ventilatory functions as they lead to a maximal expansion of the lungs, which prevents the progressive collapse of alveoli (atelectasis). Sighs also restore lung compliance and maintain normal lung function.

In some embodiments of the invention, a sigh is defined by the in vivo activity, in which a small subpopulation of neurons in a breathing control center (retrotrapezoid nucleus/parafacial respiratory group, RTN/pFRG) express neuromedin B (Nmb) or gastrin releasing peptide (Grp), activating receptors in the preBötzinger Complex (preBötC), the respiratory rhythm generator. An in vivo sigh can be monitored by the effect on breathing, for example in plethysmography traces by the characteristic biphasic ramp, the augmented flow in the second phase of the inspiratory effort and the prolongation of expiratory time following the event. Sighs may also be confirmed by visual monitoring of breathing behavior.

A sigh can also be defined by a specific signature from neurons in brain slices. Explanted preBötC brain slices show inspiratory activity detectable as rhythmic bursts of preBötC neurons and hypoglossal (cranial nerve XII) motoneuron output. A burst with two peaks (“doublet”) provides an in vitro signature of a sigh.

Bombesin-like peptides comprise a large family of peptides, initially identified from frog skin. The two known members of the bombesin family in mammals are neuromedin B (Nmb) and gastrin releasing peptide (Grp), widely distributed in mammalian neural and endocrine cells Gastrin-releasing peptide (GRP) was the first mammalian bombesin-like peptide to be characterized. The amidated decapeptide neuromedin B is the mammalian homolog of the amphibian bombesin-like peptide ranatensin. NMB is encoded in a 76-amino acid precursor. The sequence of the C-terminal decapeptide of NMB is highly conserved across mammalian species. The human complete neuromedin protein has the sequence MARRAGGARMFGSLLLFALLAAGVAPLSWDLPEPRSRASKIRVHSRGNLWATGH FMGKKSLEPSSPSHWGQLPTPPLRDQRLQLSHDLLGILLLKKALGVSLSRPAPQIQYRRLLVQIL QK, where the signal sequence is residues 1-24, the mature peptide is residues 25-57.

NMB acts by binding to its high affinity cell surface receptor, neuromedin B receptor (NMBR). This receptor is a G protein-coupled receptor with seven transmembrane spanning regions, hence the receptor is also denoted as a 7-transmembrane receptor (7-TMR). When NMB binds to NMBR (BB1), the heterotrimeric G protein that is attached to the receptor is activated. In the activated NMBR/G-protein complex, there occurs an exchange of GTP for GDP bound to G-α subunit. The G-α subunit, in turn, dissociates from the G-βγ subunits. The free G-α activates adenylate cyclase, which catalyzes the conversion of ATP to cAMP.

Gastrin-releasing peptide, also known as GRP, is a neuropeptide, a regulatory molecule that has been implicated in a number of physiological and pathophysiological processes. It is a 148-amino acid preproprotein, which is processed to produce either the 27-amino acid gastrin-releasing peptide or the 10-amino acid neuromedin C. The complete human GRP protein has the amino acid sequence MRGRELPLVL LALVLCLAPR GRAVPLPAGG GTVLTKMYPR GNHWAVGHLM GKKSTGESSS VSERGSLKQQ LREYIRWEEA ARNLLGLIEA KENRNHQPPQ PKALGNQQPS WDSEDSSNFK DVGSKGKVGR LSAPGSQREG RNPQLNQQ, where the signal sequence is amino acid residues 1-23, the mature GRP peptide is residues 24-50; and the mature neuromedin C peptide is residues 41-50.

GRP acts through gastrin-releasing peptide receptor (GRPR; BB2), a G protein-coupled receptor. This receptor is a glycosylated, 7-transmembrane G-protein coupled receptor that activates the phospholipase C signaling pathway.

“Nmb or Grp modulating agents” include molecules, i.e. ligands, that bind to and activate the NMB or GGRP receptors, which ligands may activate the receptor (agonist) or inhibit the receptor (antagonist). Included as agonists are the NMB and GRP peptides themselves, and variants thereof. Other suitable agonists or antagonists may be identified by compound screening by detecting the ability of an agent to activate the receptor. In vitro assays may be conducted as a first screen for efficacy of a candidate agent, and usually an in vivo assay will be performed to confirm the biological assay. Desirable agents are temporary in nature, e.g. due to biological degradation.

For use in the methods of the invention the NMB and GRP peptides, fusion proteins thereof, modifications thereof, or a combination of forms may be used, chemical mimetic agonists and antagonists thereof; genetic agents including siRNA, anti-sense RNA, etc.; and antibodies that the specifically bind to NMB, NMBR, GRP, or GRPR. Peptides of interest include fragments of at least about 12 contiguous amino acids, at least about 20 contiguous amino acids, at least about 30 contiguous amino acids, up to the provided mature peptide, and may extend further to comprise other sequences present in the precursor protein.

Amino acid sequence variants of the agonist peptides provided herein are also contemplated. For example, binding affinity and/or other biological properties can be improved by altering the amino acid sequence encoding the protein. Amino acids sequence variants can be prepared by introducing appropriate modifications into the nucleic acid sequence encoding the protein or by introducing the modification by peptide synthesis. Such modifications include, for example, deletions from, insertions into, and/or substitutions within the amino acid sequence. Any combination of deletion, insertion, and substitution can be made to arrive at the final amino acid construct, provided that the final construct possesses the desired characteristics. Accordingly, provided herein are variants. In some embodiments, variants comprise an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the amino acid sequence of any one of the GRP or NMB peptides, where, for example, the variant may include 1, 2, 3, 4, 5, 6, 7, 8, 9, 10 amino acid substitutions or changes relative to the provided sequence.

Modifications and changes can be made in the structure of polypeptides and proteins and still result in a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's or protein's biological functional activity, certain amino acid sequence substitutions can be made in a polypeptide or protein sequence and nevertheless obtain a polypeptide or protein with like properties.

Amino acid substitutions are generally based on the relative similarity of the amino acid side-chain substituents, for example, their hydrophobicity, hydrophilicity, charge, size, and the like. Exemplary substitutions that take one or more of the foregoing characteristics into consideration are well known to those of skill in the art and include, but are not limited to (original residue: exemplary substitution): (Ala: Gly, Ser), (Arg: Lys), (Asn: Gln, His), (Asp: Glu, Cys, Ser), (Gln: Asn), (Glu: Asp), (Gly: Ala), (His: Asn, Gln), (Ile: Leu, Val), (Leu: Ile, Val), (Lys: Arg), (Met: Leu, Tyr), (Ser: Thr), (Thr: Ser), (Tip: Tyr), (Tyr: Trp, Phe), and (Val: Ile, Leu). Embodiments of this disclosure, therefore, consider functional or biological equivalents of a polypeptide or protein as set forth above. In particular, embodiments of the polypeptides and proteins can include variants having about 50%, 60%, 70%, 80%, 90%, and 95% sequence identity to the polypeptide and protein of interest.

Modifications of interest that do not alter primary sequence include chemical derivatization of polypeptides, e.g., acetylation, or carboxylation. Also included are modifications of glycosylation, e.g. those made by modifying the glycosylation patterns of a polypeptide during its synthesis and processing or in further processing steps; e.g. by exposing the polypeptide to enzymes which affect glycosylation, such as mammalian glycosylating or deglycosylating enzymes. Also embraced are sequences that have phosphorylated amino acid residues, e.g. phosphotyrosine, phosphoserine, or phosphothreonine.

Also included in the subject invention are polypeptides that have been modified using ordinary molecular biological techniques and synthetic chemistry so as to improve their resistance to proteolytic degradation or to optimize solubility properties or to render them more suitable as a therapeutic agent. For examples, the backbone of the peptide may be cyclized to enhance stability (see Friedler et al. (2000) J. Biol. Chem. 275:23783-23789). Analogs of such polypeptides include those containing residues other than naturally occurring L-amino acids, e.g. D-amino acids or non-naturally occurring synthetic amino acids.

As an option to recombinant methods, polypeptides can be chemically synthesized. Such methods typically include solid-state approaches, but can also utilize solution based chemistries and combinations or combinations of solid-state and solution approaches. Examples of solid-state methodologies for synthesizing proteins are described by Merrifield (1964) J. Am. Chem. Soc. 85:2149; and Houghton (1985) Proc. Natl. Acad. Sci., 82:5132. Fragments of polypeptides of the invention protein can be synthesized and then joined together. Methods for conducting such reactions are described by Grant (1992) Synthetic Peptides: A User Guide, W.H. Freeman and Co., N.Y.; and in “Principles of Peptide Synthesis,” (Bodansky and Trost, ed.), Springer-Verlag, Inc. N.Y., (1993). Proteins or peptides of the invention may comprise one or more non-naturally occurring or modified amino acids. A “non-naturally occurring amino acid residue” refers to a residue, other than those naturally occurring amino acid residues listed above, which is able to covalently bind adjacent amino acid residues(s) in a polypeptide chain. Non-natural amino acids include, but are not limited to homo-lysine, homo-arginine, homo-serine, azetidinecarboxylic acid, 2-aminoadipic acid, 3-aminoadipic acid, beta-alanine, aminopropionic acid, 2-aminobutyric acid, 4-aminobutyric acid, 6-aminocaproic acid, 2-aminoheptanoic acid, 2-aminoisobutyric acid, 3-aminoisbutyric acid, 2-aminopimelic acid, tertiary-butylglycine, 2,4-diaminoisobutyric acid, desmosine, 2,2′-diaminopimelic acid, 2,3-diaminopropionic acid, N-ethylglycine, N-ethylasparagine, homoproline, hydroxylysine, allo-hydroxylysine, 3-hydroxyproline, 4-hydroxyproline, isodesmosine, allo-isoleucine, N-methylalanine, N-methylglycine, N-methylisoleucine, N-methylpentylglycine, N-methylvaline, naphthalanine, norvaline, norleucine, ornithine, citrulline, pentylglycine, pipecolic acid and thioproline. Modified amino acids include natural and non-natural amino acids which are chemically blocked, reversibly or irreversibly, or modified on their N-terminal amino group or their side chain groups, as for example, N-methylated D and L amino acids, side chain functional groups that are chemically modified to another functional group. For example, modified amino acids include methionine sulfoxide; methionine sulfone; aspartic acid- (beta-methyl ester), a modified amino acid of aspartic acid; N-ethylglycine, a modified amino acid of glycine; or alanine carboxamide and a modified amino acid of alanine. Additional non-natural and modified amino acids, and methods of incorporating them into proteins and peptides, are known in the art (see, e.g., Sandberg et al., (1998) J. Med. Chem. 41: 2481-91; Xie and Schultz (2005) Curr. Opin. Chem. Biol. 9: 548-554; Hodgson and Sanderson (2004) Chem. Soc. Rev. 33: 422-430.

If desired, various groups may be introduced into the peptide during synthesis or during expression, which allow for linking to other molecules or to a surface. Thus cysteines can be used to make thioethers, histidines for linking to a metal ion complex, carboxyl groups for forming amides or esters, amino groups for forming amides, and the like.

The polypeptides may also be isolated and purified in accordance with conventional methods of recombinant synthesis. A lysate may be prepared of the expression host and the lysate purified using HPLC, exclusion chromatography, gel electrophoresis, affinity chromatography, or other purification technique. For the most part, the compositions which are used will comprise at least 20% by weight of the desired product, more usually at least about 75% by weight, preferably at least about 95% by weight, and for therapeutic purposes, usually at least about 99.5% by weight, in relation to contaminants related to the method of preparation of the product and its purification. Usually, the percentages will be based upon total protein.

In vitro assays for sigh inducing or sigh inhibiting biological activity include, e.g. binding to the cognate receptor, activation of the receptor, including release of cyclic AMP, and biological effects. In some embodiments a screening assay includes detection of a sigh signature pattern from an in vitro slice culture comprising preBötC brain slices show inspiratory activity detectable as rhythmic bursts of preBötC neurons and hypoglossal (cranial nerve XII) motoneuron output.

A candidate agent useful as an agonist results in increased activity, e.g. cyclic AMP release, signature sigh neuronal activity, in vivo sigh activity, etc. by at least about 10%, at least about 20%, at least about 50%, at least about 70%, at least about 80%, or up to about 90% compared to level observed in absence of candidate agent.

A candidate agent useful as an antagonist results in decreased activity, e.g. cyclic AMP release, signature sigh neuronal activity, in vivo sigh activity, etc., usually detected as inhibition of activity induced by an activating agent, by at least about 10%, at least about 20%, at least about 50%, at least about 70%, at least about 80%, or up to about 90% compared to level observed in absence of candidate agent.

Sigh antagonists. Agents of interest as inhibitors of sighs include specific binding members that inhibit the signaling activity of either of NMBR and GRPR. The term “specific binding member” or “binding member” as used herein refers to a member of a specific binding pair, i.e. two molecules, usually two different molecules, where one of the molecules (i.e., first specific binding member) through chemical or physical means specifically binds to the other molecule (i.e., second specific binding member). Inhibitors useful in the methods of the invention include analogs, derivatives and fragments of the original specific binding member.

In some embodiments, the specific binding member is an antibody. The term “antibody” or “antibody moiety” is intended to include any polypeptide chain-containing molecular structure with a specific shape that fits to and recognizes an epitope, where one or more non-covalent binding interactions stabilize the complex between the molecular structure and the epitope. Antibodies utilized in the present invention may be polyclonal antibodies, although monoclonal antibodies are preferred because they may be reproduced by cell culture or recombinantly, and can be modified to reduce their antigenicity.

Polyclonal antibodies can be raised by a standard protocol by injecting a production animal with an antigenic composition. See, e.g., Harlow and Lane, Antibodies: A Laboratory Manual, Cold Spring Harbor Laboratory, 1988. Alternatively, for monoclonal antibodies, hybridomas may be formed by isolating the stimulated immune cells, such as those from the spleen of the inoculated animal. These cells are then fused to immortalized cells, such as myeloma cells or transformed cells, which are capable of replicating indefinitely in cell culture, thereby producing an immortal, immunoglobulin-secreting cell line. In addition, the antibodies or antigen binding fragments may be produced by genetic engineering. Humanized, chimeric, or xenogeneic human antibodies, which produce less of an immune response when administered to humans, are preferred for use in the present invention.

In addition to entire immunoglobulins (or their recombinant counterparts), immunoglobulin fragments comprising the epitope binding site (e.g., Fab′, F(ab′)₂, or other fragments) are useful as antibody moieties in the present invention. Such antibody fragments may be generated from whole immunoglobulins by ricin, pepsin, papain, or other protease cleavage. “Fragment,” or minimal immunoglobulins may be designed utilizing recombinant immunoglobulin techniques. For instance “Fv” immunoglobulins for use in the present invention may be produced by linking a variable light chain region to a variable heavy chain region via a peptide linker (e.g., poly-glycine or another sequence which does not form an alpha helix or beta sheet motif).

In one embodiment of the invention, the agent, or a pharmaceutical composition comprising the agent, is provided in an amount effective to detectably modulate ventilation. The effective amount is determined via empirical testing routine in the art.

An “individual” is a vertebrate, preferably a mammal, more preferably a human. Mammals include, but are not limited to, rodents, primates, farm animals, sport animals, and pets. The terms “recipient”, “individual”, “subject”, “host”, and “patient”, used interchangeably herein and refer to any mammalian subject for whom diagnosis, treatment, or therapy is desired, particularly humans.

An “effective amount” is an amount sufficient to effect beneficial or desired clinical results. An effective amount can be administered in one or more administrations. For purposes of this invention, an effective amount of an inhibitor or agonist of *** is an amount that is sufficient to palliate, ameliorate, stabilize, reverse, slow or delay the progression of the disease state by modulating breathing of the subject.

As used herein, “treatment” is an approach for obtaining beneficial or desired clinical results. For purposes of this invention, beneficial or desired clinical results include, but are not limited to, alleviation of symptoms, diminishment of extent of disease, stabilized (i.e., not worsening) state of disease, preventing spread (i.e., metastasis) of disease, delay or slowing of disease progression, amelioration or palliation of the disease state, and remission (whether partial or total), whether detectable or undetectable. “Treatment” can also mean prolonging survival as compared to expected survival if not receiving treatment. “Palliating” a disease means that the extent and/or undesirable clinical manifestations of a disease state are lessened and/or time course of the progression is slowed or lengthened, as compared to not administering the methods of the present invention.

“Therapeutic target” refers to a gene or gene product that, upon modulation of its activity (e.g., by modulation of expression, biological activity, and the like), can provide for modulation of a phenotype. As used throughout, “modulation” is meant to refer to an increase or a decrease in the indicated phenomenon (e.g., modulation of a biological activity refers to an increase in a biological activity or a decrease in a biological activity).

### Methods of Use

Methods are provided for the modulation of sigh breathing in an individual, by contacting cells of the breathing control center, i.e. the preBötC region, with an effective dose of an agent that agonizes or antagonizes activity of one or both the neuromedin B or gastrin releasing peptide receptors. The effective dose of an agonist will activate a pattern of sighs. The effective dose of an antagonist will decrease a pattern of sighs. Such methods include administering to a subject in need of treatment a therapeutically effective amount or an effective dose of the agents of the invention, including without limitation combinations of the agent with an additional therapy. The therapy may be systemic, or may be targeted to the brain.

In some of these embodiments, the therapeutically effective dose is administered once daily. In some embodiments administered twice daily. In some embodiments administered 3 times day. In some embodiments administered 4 times a day, or more. In some of these embodiments, the therapeutically effective dose is administered on consecutive days for at least a week, at least a month, at least a year, or on as needed basis for the rest of the patient's life. The therapeutically effective dose can be about 0.1 to about 10,000 mg/day.

In other aspects, the compounds provided by the invention are used in the manufacture of a medicament for the modulation of breathing, wherein said medicament is an agonist or antagonist of NMBR or GRPR. In various embodiments, the medicament is formulated for administration, including both immediate release and sustained release pharmaceutical formulations. In all of these embodiments, the invention provides unit dose forms of the medicament.

In another aspect, the present invention provides methods for selecting patients likely to benefit from the therapies of the invention, as well as methods for determining whether a patient is responding to such therapy. In these methods, breathing analysis is determined and compared to a control value.

In some embodiments of the invention, an inhibitor of sigh breathing is administered to an individual in need thereof. Conditions of hyperventilation include certain disturbances in cardiorespiratory coupling characteristic of familial dysautonomia and sickle-cell anemia. In sickle-cell anemia, the frequency of sighs is not different from controls, but sighs are much more likely to induce pronounced perfusion drops that are may be associated with an exaggerated sympathetic and suppressed parasympathetic response. Hypoperfusion in sickle-cell anemia is a particularly dangerous situation, because it could lead to red blood cell polymerization followed by a vaso-occlusive crisis. Indeed, the sigh-triggered hypoperfusion may be responsible for the sudden death that occurs frequently in this patient population.

Patients with panic disorder suffer from significantly increased respiratory variability and hyperventilation. Their breathing irregularity persists even when panic is pharmacologically controlled, indicating that respiratory dysregulation is not secondarily induced by the panic attack but may convey panic attack vulnerability. Excessive random breathing variability in panic disorder patients may centrally activate sighs possibly in a physiological attempt to reduce and reset this pathological variability. Indeed, patients with panic disorders typically show increased sigh frequency. However, instead of regularizing variability as is the case in healthy subjects, the increased generation of the sighs may exaggerate the already existing breathing variability by further increasing tidal volume variability, which is a characteristic feature of patients with panic disorder. Sighs could further increase irregularity through the so-called postsigh apnea, which can create conditions of intermittent hypoxia and oxidative stress if sighs occur frequently.

Included as a condition for treatment is chronic or acute hyperventilation syndrome (HVS). HVS is a condition in which minute ventilation exceeds metabolic demands, resulting in hemodynamic and chemical changes that produce characteristic dysphoric symptoms. Symptoms of HVS and panic disorder overlap considerably, though the two conditions remain distinct. Approximately 50% of patients with panic disorder and 60% of patients with agoraphobia manifest hyperventilation as a symptom, whereas 25% of patients with HVS manifest panic disorder.

HVS occurs in acute and chronic forms. Acute HVS accounts for only 1% of cases but is more easily diagnosed. Chronic HVS can present with a myriad of respiratory, cardiac, neurologic, or gastrointestinal (GI) symptoms without any clinically apparent overbreathing by the patient. Hypocapnia can be maintained without any overt change in the minute ventilation if the patient exhibits frequent sighs interspersed with normal respirations. The underlying mechanism by which some patients develop hyperventilation is unknown. One theory suggests that certain stressors provoke an exaggerated respiratory response.

In most patients, the mechanics of breathing are disordered in a characteristic way. When stressed, these patients rely on thoracic breathing rather than diaphragmatic breathing, resulting in a hyperexpanded chest and high residual lung volume. Because of the high residual volume, they are then unable to take a normal tidal volume with the next breath and consequently experience dyspnea.

Clearly, HVS not only causes severe and genuine discomfort for the patient but also accounts for considerable medical expense through the process of excluding more serious disorders. The chronicity of the condition often causes different physicians to repeat these diagnostic investigations.

Such patients may be treated acutely with an inhibitor of NMDR or GRPR for a period of time sufficient to stabilize regular breathing.

Conditions in which an agonist of sigh breathing may be administered include without limitation congenital hypoventilation syndrome (CCHS), children with sleep-disordered breathing, including those at risk of sudden infant death syndrome (SIDS). The sigh may also have an important role in obstructive sleep apnea, specifically during the recovery from an airway obstruction. The termination of airway occlusion is typically abrupt and associated with a sudden burst of genioglossus activity and the recruitment of phasic inspiratory motor units. Reflex recruitment of pharyngeal dilator muscles seems to be insufficient for this abrupt response, and central mechanisms that stage the initiation of arousal become important.

Enhanced sigh breathing can also be advantageous post-surgery, and in combination with an artificial respirator, e.g. patients that are fully ventilated, or paralyzed. For example the addition of one sigh per minute during surgical or post-surgical ventilation can improve arterial oxygenation and promote alveolar recruitment. The dose may be used to increase sigh breathing to a level of from about 1 to 3 sighs per minute, or may be one sigh per two minutes, three minutes, four minutes, five minutes, and the like. Partial ventilatory techniques prevent paralysis and reduce the use of sedative drugs. By adding sighs it may be possible to extend the indication for the use of partial ventilatory techniques.

### Screening Methods

The methods of the invention include screening compounds that mimic or increase activity of NMBR or GRPR and modulate sigh responses. Such screening methods may include, for example, a step of determining activity in a biological setting, e.g. increase or decrease of sighs in breathing. In these embodiments the control peptides, NMB or GRP, are used to establish a target sigh signature in an in vitro culture system, where candidate agents are tested for the ability to mimic the signature. In some embodiments the structure of the NMB or GRP peptide is utilized as a starting point for rational drug design. Drug screening identifies agents that mimic, agonize or antagonize sigh inducing activity. A wide variety of assays may be used for this purpose, including labeled in vitro protein-protein binding assays, electrophoretic mobility shift assays, immunoassays for protein binding, and the like. Knowledge of the 3-dimensional structure leads to the rational design of small drugs that specifically mimic their activity.

Candidate agents of interest are biologically active agents that encompass numerous chemical classes, primarily organic molecules, which may include organometallic molecules, inorganic molecules, genetic sequences, etc. An important aspect of the invention is to evaluate candidate drugs, select therapeutic antibodies and protein-based therapeutics, with preferred biological response functions. Candidate agents comprise functional groups necessary for structural interaction with proteins, particularly hydrogen bonding, and typically include at least an amine, carbonyl, hydroxyl or carboxyl group, frequently at least two of the functional chemical groups. The candidate agents often comprise cyclical carbon or heterocyclic structures and/or aromatic or polyaromatic structures substituted with one or more of the above functional groups. Candidate agents are also found among biomolecules, including peptides, polynucleotides, saccharides, fatty acids, steroids, purines, pyrimidines, derivatives, structural analogs or combinations thereof.

Included are pharmacologically active drugs, genetically active molecules, etc. Compounds of interest include chemotherapeutic agents, anti-inflammatory agents, hormones or hormone antagonists, ion channel modifiers, and neuroactive agents. Exemplary of pharmaceutical agents suitable for this invention are those described in, “The Pharmacological Basis of Therapeutics,” Goodman and Gilman, McGraw-Hill, New York, N.Y., (1996), Ninth edition, under the sections: Drugs Acting at Synaptic and Neuroeffector Junctional Sites; Drugs Acting on the Central Nervous System; Autacoids: Drug Therapy of Inflammation; Water, Salts and Ions; Drugs Affecting Renal Function and Electrolyte Metabolism; Cardiovascular Drugs; Drugs Affecting Gastrointestinal Function; Drugs Affecting Uterine Motility; Chemotherapy of Parasitic Infections; Chemotherapy of Microbial Diseases; Chemotherapy of Neoplastic Diseases; Drugs Used for Immunosuppression; Drugs Acting on Blood-Forming organs; Hormones and Hormone Antagonists; Vitamins, Dermatology; and Toxicology, all incorporated herein by reference.

Test compounds include all of the classes of molecules described above, and may further comprise samples of unknown content. Of interest are complex mixtures of naturally occurring compounds derived from natural sources such as plants. While many samples will comprise compounds in solution, solid samples that can be dissolved in a suitable solvent may also be assayed. Samples of interest include environmental samples, e.g. ground water, sea water, mining waste, etc.; biological samples, e.g. lysates prepared from crops, tissue samples, etc.; manufacturing samples, e.g. time course during preparation of pharmaceuticals; as well as libraries of compounds prepared for analysis; and the like. Samples of interest include compounds being assessed for potential therapeutic value, i.e. drug candidates.

The term samples also includes the fluids described above to which additional components have been added, for example components that affect the ionic strength, pH, total protein concentration, etc. In addition, the samples may be treated to achieve at least partial fractionation or concentration. Biological samples may be stored if care is taken to reduce degradation of the compound, e.g. under nitrogen, frozen, or a combination thereof. The volume of sample used is sufficient to allow for measurable detection, usually from about 0.1 μl to 1 ml of a biological sample is sufficient.

Compounds, including candidate agents, are obtained from a wide variety of sources including libraries of synthetic or natural compounds. For example, numerous means are available for random and directed synthesis of a wide variety of organic compounds, including biomolecules, including expression of randomized oligonucleotides and oligopeptides. Alternatively, libraries of natural compounds in the form of bacterial, fungal, plant and animal extracts are available or readily produced. Additionally, natural or synthetically produced libraries and compounds are readily modified through conventional chemical, physical and biochemical means, and may be used to produce combinatorial libraries. Known pharmacological agents may be subjected to directed or random chemical modifications, such as acylation, alkylation, esterification, amidification, etc. to produce structural analogs.

Agents are screened for biological activity by adding the agent to at least one and usually a plurality of assay conditions, for example in conjunction with assay conditions where the agent is not present, conditions where a known activator are present, conditions in which a known activity is expected, e.g. cyclic AMP release, and the like. The change in parameter readout in response to the agent is measured, desirably normalized, and the resulting data may then be evaluated by comparison to reference data.

The agents are conveniently added in solution, or readily soluble form, to the medium of cells in culture. The agents may be added in a flow-through system, as a stream, intermittent or continuous, or alternatively, adding a bolus of the compound, singly or incrementally, to an otherwise static solution. In a flow-through system, two fluids are used, where one is a physiologically neutral solution, and the other is the same solution with the test compound added. The first fluid is passed over the cells, followed by the second. In a single solution method, a bolus of the test compound is added to the volume of medium surrounding the cells. The overall concentrations of the components of the culture medium should not change significantly with the addition of the bolus, or between the two solutions in a flow through method.

Preferred agent formulations do not include additional components, such as preservatives, that may have a significant effect on the overall formulation. Thus preferred formulations consist essentially of a biologically active compound and a physiologically acceptable carrier, e.g. water, ethanol, DMSO, etc. However, if a compound is liquid without a solvent, the formulation may consist essentially of the compound itself.

A plurality of assays may be run in parallel with different agent concentrations to obtain a differential response to the various concentrations. As known in the art, determining the effective concentration of an agent typically uses a range of concentrations resulting from 1:10, or other log scale, dilutions. The concentrations may be further refined with a second series of dilutions, if necessary. Typically, one of these concentrations serves as a negative control, i.e. at zero concentration or below the level of detection of the agent or at or below the concentration of agent that does not give a detectable change in the phenotype.

The compounds having the desired pharmacological activity may be administered in a physiologically acceptable carrier to a host for modulation of breathing, etc. The agents may be administered in a variety of ways, orally, by inhalation, topically, parenterally e.g. subcutaneously, intraperitoneally, by viral infection, intravascularly, etc. Depending upon the manner of introduction, the compounds may be formulated in a variety of ways. The concentration of therapeutically active compound in the formulation may vary from about 0.1-10 wt %.

Binding assays may be performed conventionally. Biological assays, e.g. detection of a sigh signature in an in vitro brain slice, may be generated from a biological sample using any convenient protocol, for example as described in the examples. The readout may be a mean, average, median or the variance or other statistically or mathematically-derived value associated with the measurement. A signature pattern may be evaluated on a number of points: to determine if there is a statistically significant change at any point in the data matrix; whether the change is an increase or decrease in the motor neuron output; whether the change is specific for one or more physiological states or factors, and the like.

Following obtainment of the signature pattern from the sample being assayed, the signature pattern is compared with a reference or control profile to make an evaluation regarding activity of a candidate agent, and the like. Typically a comparison is made with a sample or set of samples from a control profile.

In order to identify profiles that are indicative of responsiveness, a statistical test will provide a confidence level for a change in the expression, titers or concentration of markers between the test and control profiles to be considered significant, where the control profile may be for responsiveness or non-responsiveness. The raw data may be initially analyzed by measuring the values for each marker, usually in duplicate, triplicate, quadruplicate or in 5-10 replicate features per marker.

The analysis and database storage may be implemented in hardware or software, or a combination of both. In one embodiment of the invention, a machine-readable storage medium is provided, the medium comprising a data storage material encoded with machine readable data which, when using a machine programmed with instructions for using said data, is capable of displaying a any of the datasets and data comparisons of this invention. Such data may be used for a variety of purposes, such as patient monitoring, initial diagnosis, and the like. Preferably, the invention is implemented in computer programs executing on programmable computers, comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer may be, for example, a personal computer, microcomputer, or workstation of conventional design.

Each program is preferably implemented in a high level procedural or object oriented programming language to communicate with a computer system. However, the programs can be implemented in assembly or machine language, if desired. In any case, the language may be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system may also be considered to be implemented as a computer-readable storage medium, configured with a computer program, where the storage medium so configured causes a computer to operate in a specific and predefined manner to perform the functions described herein.

A variety of structural formats for the input and output means can be used to input and output the information in the computer-based systems of the present invention. One format for an output means test datasets possessing varying degrees of similarity to a trusted profile. Such presentation provides a skilled artisan with a ranking of similarities and identifies the degree of similarity contained in the test pattern.

The signature patterns and databases thereof may be provided in a variety of media to facilitate their use. “Media” refers to a manufacture that contains the signature pattern information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure may be chosen, based on the means used to access the stored information. A variety of data processor programs and formats can be used for storage, e.g. word processing text file, database format, etc.

### Pharmaceutical Formulations

A “delayed release dosage form” is one that releases a drug (or drugs) at a time other than promptly after administration.

An “extended release dosage form” is one that allows at least a twofold reduction in dosing frequency as compared to the drug presented as a conventional dosage form (e.g. as a solution or prompt drug-releasing, conventional solid dosage form).

A “pulsatile release dosage form” is one that mimics a multiple dosing profile without repeated dosing and allows at least a twofold reduction in dosing frequency as compared to that drug presented as a conventional dosage form (e.g. as a solution or prompt drug-releasing, conventional solid dosage form). A pulsatile release profile is characterized by a time period of no release (lag time) followed by rapid drug release.

A “modified release dosage form” is one for which the drug release characteristics of time, course and/or location are chosen to accomplish therapeutic or convenience objectives not offered by conventional dosage forms such as solutions, ointments, or promptly dissolving dosage forms. Delayed release and extended release dosage forms and their combinations are types of modified release dosage forms. The pharmaceutical combination of the invention may have any or all of its constituents in a modified release dosage form. A “modified release pharmaceutical composition” has at least one of its components in modified release dosage form.

As used herein “active compounds” in addition to their free base and quaternized forms also encompasses pharmaceutically acceptable, pharmacologically active derivatives of active compounds including individual enantiomers and their pharmaceutically acceptable salts, mixtures of enantiomers and their pharmaceutically acceptable salts, and active metabolites of active compounds and their pharmaceutically acceptable salts, unless otherwise noted. It is understood that in some cases dosages of enantiomers, derivatives, and metabolites may need to be adjusted based on relative activity of the racemic mixture of active compound.

As used herein, “pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxyrnaleic, phenylacetic, glutamic, benzoic, salicylic, sulfanilic, 2-acetoxybenzoic, fumaric, toluenesulfonic, methanesulfonic, ethane disulfonic, oxalic, and isethionic.

The pharmaceutically acceptable salts of the compounds can be synthesized from the parent compound, which contains a basic or acidic moiety, by conventional chemical methods.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

As used herein, the term “stereoisomers” refers to compounds made up of the same atoms bonded by the same bonds but having different spatial structures which are not interchangeable. The three-dimensional structures are called configurations. As used herein, the term “enantiomers” refers to two stereoisomers whose molecules are nonsuperimposable mirror images of one another. As used herein, the term “optical isomer” is equivalent to the term “enantiomer”. The terms “racemate”, “racemic mixture” or “racemic modification” refer to a mixture of equal parts of enantiomers. The term “chiral center” refers to a carbon atom to which four different groups are attached. The term “enantiomeric enrichment” as used herein refers to the increase in the amount of one enantiomer as compared to the other. Enantiomeric enrichment is readily determined by one of ordinary skill in the art using standard techniques and procedures, such as gas or high performance liquid chromatography with a chiral column. Choice of the appropriate chiral column, eluent and conditions necessary to effect separation of the enantiomeric pair is well within the knowledge of one of ordinary skill in the art using standard techniques well known in the art, such as those described by J. Jacques, et al., “Enantiomers, Racemates, and Resolutions”, John Wley and Sons, Inc., 1981. Examples of resolutions include recrystallization of diastereomeric salts/derivatives or preparative chiral chromatography.

The pharmaceutical compositions of the invention can be administered adjunctively with other active compounds such as analgesics, anti-inflammatory drugs, antipyretics, antidepressants, antiepileptics, antihistamines, antimigraine drugs, antimuscarinics, anxioltyics, sedatives, hypnotics, antipsychotics, bronchodilators, anti asthma drugs, cardiovascular drugs, corticosteroids, dopaminergics, electrolytes, gastrointestinal drugs, muscle relaxants, nutritional agents, vitamins, parasympathomimetics, stimulants, anorectics and anti-narcoleptics.

By adjunctive administration is meant simultaneous administration of the compounds, in the same dosage form, simultaneous administration in separate dosage forms, and separate administration of the compounds.

Formulations are prepared using a pharmaceutically acceptable “carrier” composed of materials that are considered safe and effective and may be administered to an individual without causing undesirable biological side effects or unwanted interactions. The “carrier” is all components present in the pharmaceutical formulation other than the active ingredient or ingredients. The term “carrier” includes but is not limited to diluents, binders, lubricants, disintegrators, fillers, and coating compositions.

“Carrier” also includes all components of the coating composition which may include plasticizers, pigments, colorants, stabilizing agents, and glidants. The delayed release dosage formulations may be prepared as described in references such as “Pharmaceutical dosage form tablets”, eds. Liberman et. al. (New York, Marcel Dekker, Inc., 1989), “Remington—The science and practice of pharmacy”, 20th ed., Lippincott Williams & Wilkins, Baltimore, Md., 2000, and “Pharmaceutical dosage forms and drug delivery systems”, .sup.6th Edition, Ansel et. al., (Media, Pa.: Williams and Wilkins, 1995) which provides information on carriers, materials, equipment and process for preparing tablets and capsules and delayed release dosage forms of tablets, capsules, and granules.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name Eudragit® (Roth Pharma, Westerstadt, Germany), Zein, shellac, and polysaccharides.

Additionally, the coating material may contain conventional carriers such as plasticizers, pigments, colorants, glidants, stabilization agents, pore formers and surfactants.

Optional pharmaceutically acceptable excipients present in the drug-containing tablets, beads, granules or particles include, but are not limited to, diluents, binders, lubricants, disintegrants, colorants, stabilizers, and surfactants. Diluents, also termed “fillers,” are typically necessary to increase the bulk of a solid dosage form so that a practical size is provided for compression of tablets or formation of beads and granules. Suitable diluents include, but are not limited to, dicalcium phosphate dihydrate, calcium sulfate, lactose, sucrose, mannitol, sorbitol, cellulose, microcrystalline cellulose, kaolin, sodium chloride, dry starch, hydrolyzed starches, pregelatinized starch, silicone dioxide, titanium oxide, magnesium aluminum silicate and powder sugar.

Binders are used to impart cohesive qualities to a solid dosage formulation, and thus ensure that a tablet or bead or granule remains intact after the formation of the dosage forms. Suitable binder materials include, but are not limited to, starch, pregelatinized starch, gelatin, sugars (including sucrose, glucose, dextrose, lactose and sorbitol), polyethylene glycol, waxes, natural and synthetic gums such as acacia, tragacanth, sodium alginate, cellulose, including hydroxypropylmethylcellulose, hydroxypropylcellulose, ethylcellulose, and veegum, and synthetic polymers such as acrylic acid and methacrylic acid copolymers, methacrylic acid copolymers, methyl methacrylate copolymers, aminoalkyl methacrylate copolymers, polyacrylic acid/polymethacrylic acid and polyvinylpyrrolidone.

Lubricants are used to facilitate tablet manufacture. Examples of suitable lubricants include, but are not limited to, magnesium stearate, calcium stearate, stearic acid, glycerol behenate, polyethylene glycol, talc, and mineral oil.

Disintegrants are used to facilitate dosage form disintegration or “breakup” after administration, and generally include, but are not limited to, starch, sodium starch glycolate, sodium carboxymethyl starch, sodium carboxymethylcellulose, hydroxypropyl cellulose, pregelatinized starch, clays, cellulose, alginine, gums or cross linked polymers, such as cross-linked PVP (Polyplasdone XL from GAF Chemical Corp).

Stabilizers are used to inhibit or retard drug decomposition reactions which include, by way of example, oxidative reactions.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

If desired, the tablets, beads granules or particles may also contain minor amount of nontoxic auxiliary substances such as wetting or emulsifying agents, dyes, pH buffering agents, and preservatives.

The immediate release dosage unit of the dosage form—i.e., a tablet, a plurality of drug-containing beads, granules or particles, or an outer layer of a coated core dosage form—contains a therapeutically effective quantity of the active agent with conventional pharmaceutical excipients. The immediate release dosage unit may or may not be coated, and may or may not be admixed with the delayed release dosage unit or units (as in an encapsulated mixture of immediate release drug-containing granules, particles or beads and delayed release drug-containing granules or beads).

Each dosage form contains a therapeutically effective amount of active agent. The effective dose may be from about 0.1 μg to about 1 g/kg weight of the subject. For dosage forms that mimic the twice daily dosing profile, approximately 30 wt. % to 80 wt. %, preferably 40 wt. % to 70 wt. %, of the total amount of active agent in the dosage form is released in the initial pulse, and, correspondingly approximately 70 wt. % to 20 wt. %, preferably 60 wt. % to 30 wt. %, of the total amount of active agent in the dosage form is released in the second pulse. For dosage forms mimicking the twice daily dosing profile, the second pulse is preferably released approximately 3 hours to less than 14 hours, and most preferably approximately 5 hours to 12 hours, following administration.

A kit is provided wherein the pharmaceutical composition of the invention is packaged accompanied by instructions. The packaging material may be a box, bottle, blister package, tray, or card. The kit will include a package insert instructing the patient to take a specific dose at a specific time, for example, a first dose on day one, a second dose on day two, a third dose on day three, and so on, until a maintenance dose is reached.

As will be appreciated by those skilled in the art and as described in the pertinent texts and literature, a number of methods are available for preparing drug-containing tablets, beads, granules or particles that provide a variety of drug release profiles. Such methods include, but are not limited to, the following: coating a drug or drug-containing composition with an appropriate coating material.

The methods illustrated in this disclosure are not intended to be exclusive of other methods within the scope of the present subject matter. Those of ordinary skill in the art will understand, upon reading and comprehending this disclosure, other methods within the scope of the present subject matter. The above-identified embodiments, and portions of the illustrated embodiments, are not necessarily mutually exclusive. These embodiments, or portions thereof, can be combined. In various embodiments, the methods provided above are implemented as a computer data signal embodied in a carrier wave or propagated signal, that represents a sequence of instructions which, when executed by a processor cause the processor to perform the respective method. In various embodiments, methods provided above are implemented as a set of instructions contained on a computer-accessible medium capable of directing a processor to perform the respective method. In various embodiments, the medium is a magnetic medium, an electronic medium, or an optical medium.

Although specific embodiments have been illustrated and described herein, it will be appreciated by those of ordinary skill in the art that any arrangement which is calculated to achieve the same purpose may be substituted for the specific embodiment shown. This application is intended to cover adaptations or variations of the present subject matter. It is to be understood that the above description is intended to be illustrative, and not restrictive. Combinations of the above embodiments as well as combinations of portions of the above embodiments in other embodiments will be apparent to those of skill in the art upon reviewing the above description. The scope of the present subject matter should be determined with reference to the appended claims, along with the full scope of equivalents to which such claims are entitled.

### Example 1

### The Peptidergic Control Circuit for Sighing

Sighs are long, deep breaths expressing sadness, relief, or exhaustion. Sighs also occur spontaneously every few minutes to reinflate alveoli, and sighing increases under hypoxia, stress, and certain psychiatric conditions. Here we use molecular, genetic, and pharmacologic approaches to identify a peptidergic sigh control circuit in murine brain. Small neural subpopulations in a key breathing control center (RTN/pFRG) express bombesin-like neuropeptide genes neuromedin B (Nmb) or gastrin releasing peptide (Grp). These project to the preBötzinger Complex (preBötC), the respiratory rhythm generator, which expresses NMB and GRP receptors in overlapping subsets of ˜200 neurons. Introducing either neuropeptide into preBötC, or onto preBötC slices, induced sighing, whereas elimination or inhibition of either receptor reduced basal sighing and inhibition of both abolished it. Ablating receptor-expressing neurons eliminated basal and hypoxia-induced sighing, but left breathing otherwise intact initially. We propose these overlapping peptidergic pathways comprise the core of a sigh control circuit that integrates physiological and perhaps emotional input to transform normal breaths into sighs.

We tested the effect of injecting bombesin into preBötC, and we screened for genes selectively expressed in breathing control centers. These parallel approaches led to identification of two bombesin-like neuropeptide pathways connecting the retrotrapezoid nucleus/parafacial respiratory group (RTN/pFRG), another medullary breathing control center, to preBötC. We provide genetic, pharmacologic and neural ablation evidence that these pathways are critical endogenous regulators of sighing and define the core of a dedicated sigh control circuit.

### Results

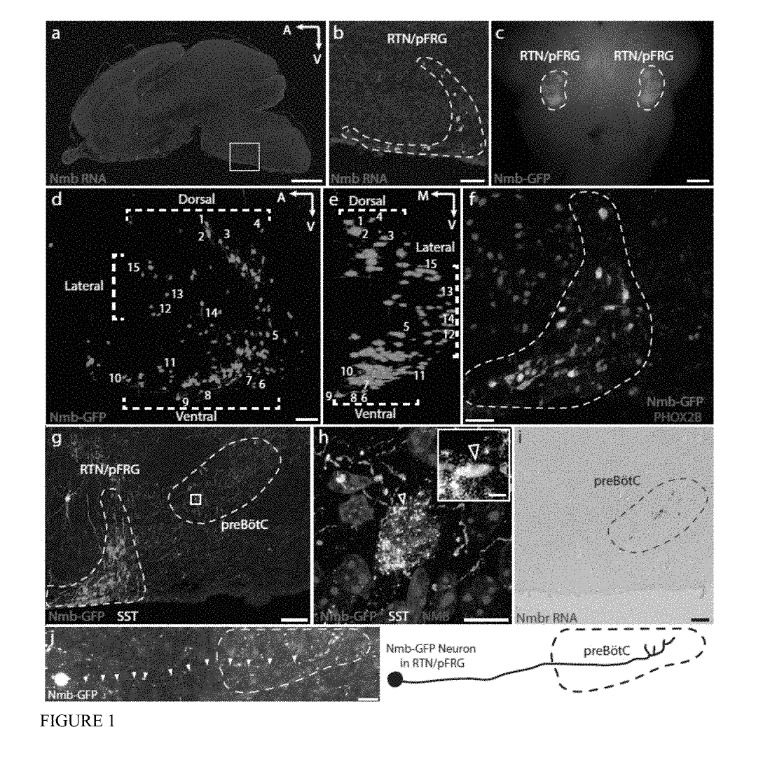

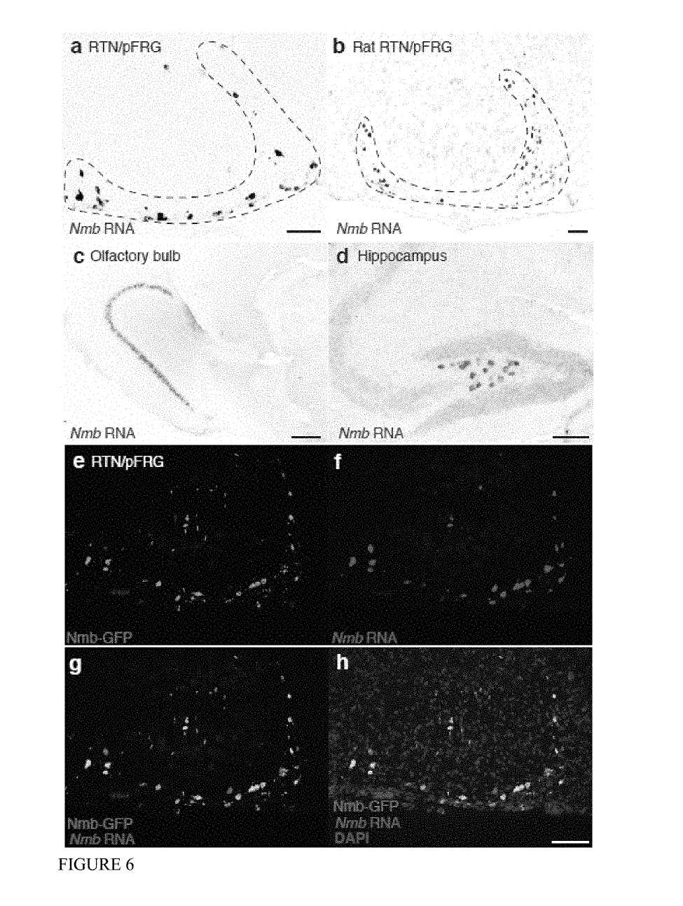

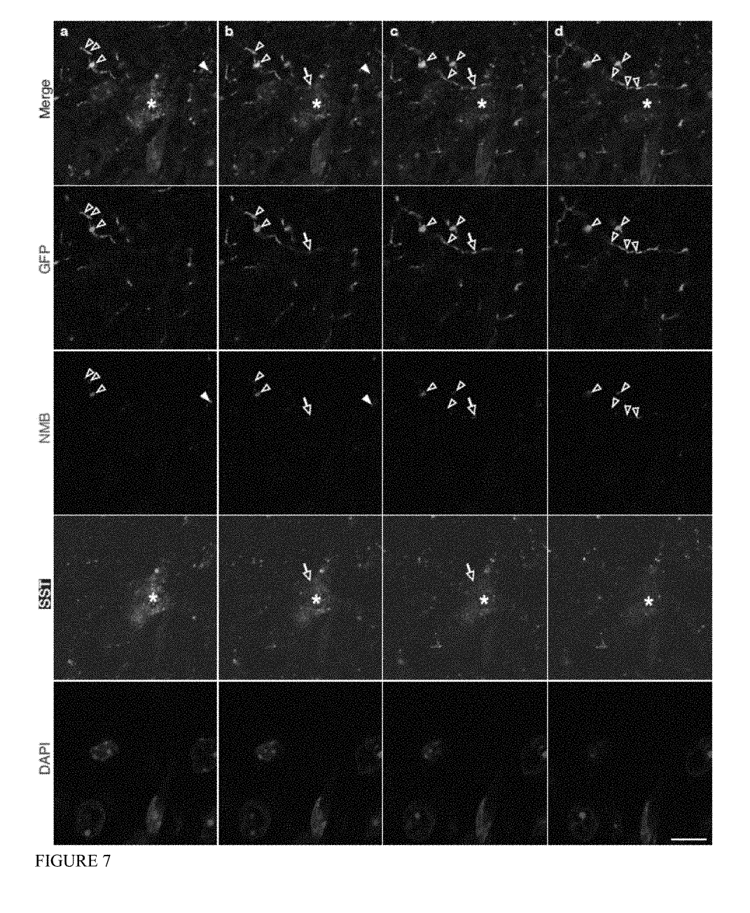

Neuromedin B links two breathing control centers To identify breathing control genes, we screened >19,000 gene expression patterns in embryonic day 14.5 mouse hindbrain. The most specific pattern was neuromedin B (Nmb), one of two genes encoding bombesin-like neuropeptides in mammals. Nmb is expressed in the medulla surrounding the lateral half of the facial nucleus, in or near RTN/pFRG in mouse (FIG. 1a,b) and rat (FIG. 6a,b). Nmb mRNA was also detected in olfactory bulb and hippocampus (FIG. 6c,d). Nmb expression was further characterized using an Nmb-GFP BAC transgene, which reproduced the endogenous Nmb pattern (FIG. 1b, c). Nmb-GFP expressed in 206±21 (mean±S.D., n=4) RTN/pFRG neurons per side, most of which (92%, n=53 cells scored) co-expressed Nmb mRNA (FIG. 6e-h). In CLARITY-processed brainstems, GFP-labeled cells surrounded the lateral half of the facial nucleus, with highest density ventral and dorsal (FIG. 1d,e). This ventral parafacial region is the RTN, an important sensory integration center for breathing. Nearly all Nmb-GFP-positive cells (96%; n=202 cells from 2 animals) co-expressed canonical RTN marker PHOX2B (FIG. 1f), comprising one-fourth of the ˜800 PHOX2B-positive RTN neurons. Nmb-expressing neurons projected to preBötC (FIG. 1g,j). Punctate NMB staining was detected along the projections (FIG. 7), some puncta abutting somatostatin-positive preBötC neurons (FIG. 1h; FIG. 7). ˜90 preBötC neurons expressed Nmbr, the GPCR specific for NMB (FIG. 1i, see below). Thus, Nmb-expressing RTN/pFRG neurons may directly modulate preBötC neurons.

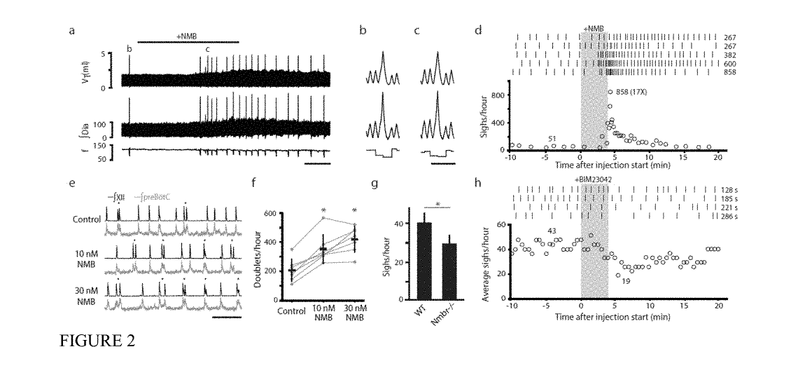

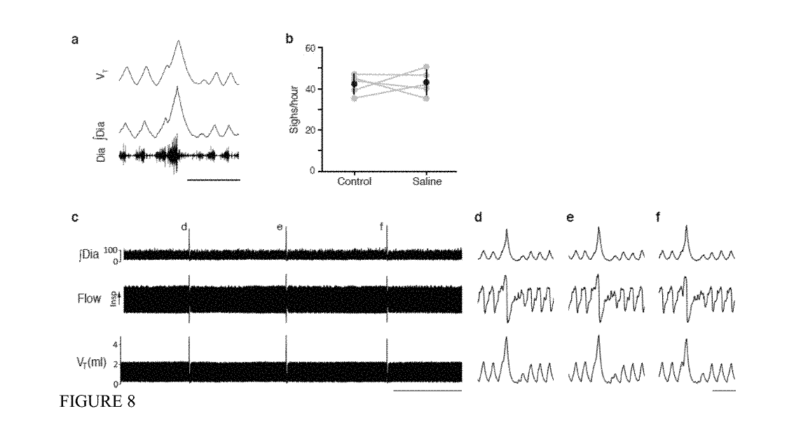

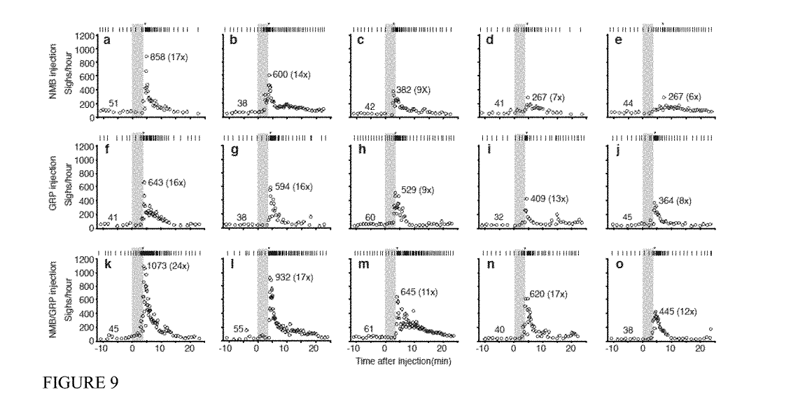

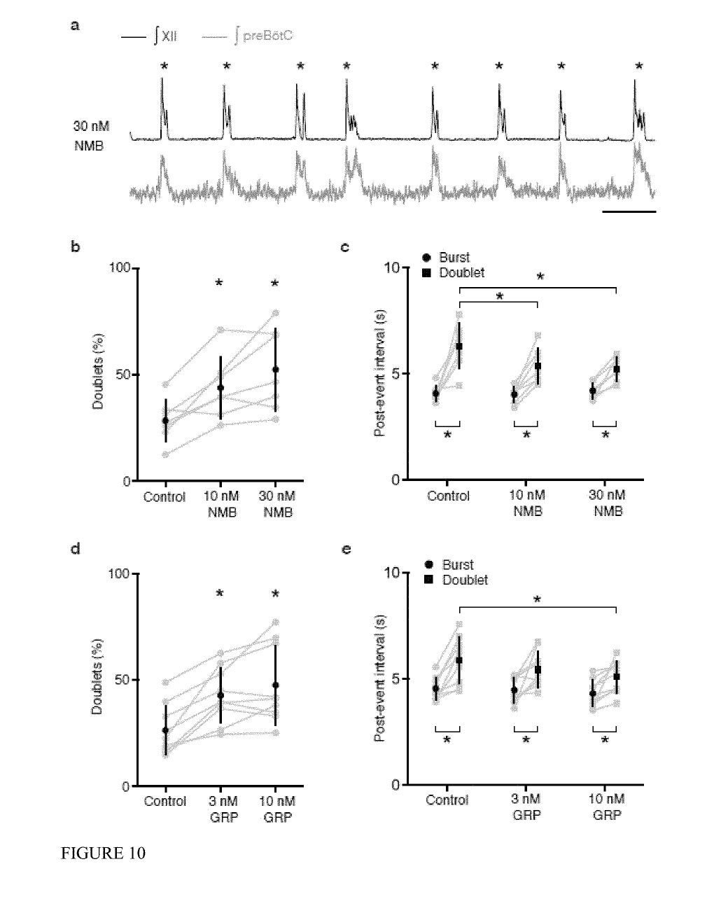

NMB injection into preBötC induces sighing. To investigate function of this NMB pathway, the peptide was microinjected into preBötC of urethane-anesthetized adult rats. Before injection, and after control (saline) injections, airflow and diaphragmatic activity (DIA_{EMG}) showed the normal (eupneic) breathing pattern, with diaphragm activity bursts during inspiration (FIG. 2a, FIG. 8a). Every minute or two, we observed a sigh (44±10/hour, n=24; unaffected by saline injection, FIG. 8b), a biphasic double-sized breath coincident with a bimodal DIA_{EMG} event (FIG. 2c and FIG. 8a,c-f). Amplitude and timescale of the first component of a sigh was indistinguishable from eupneic breaths, like human sighs. Following bilateral NMB microinjection (100 nl, 3 μM) into preBötC, sighing increased 6-17-fold (n=5; FIG. 2d, FIG. 9a-e). Effect peaked several minutes after injection and persisted 10-15 minutes afterwards. We also tested NMB on explanted preBötC brain slices of neonatal mice, where inspiratory activity is detected as rhythmic bursts of preBötC neurons and hypoglossal (cranial nerve XII) motoneuron output (FIG. 2e). Occasionally, a burst with two peaks (“doublet”) was observed (FIG. 2e)₁₁, a proposed in vitro signature of a sigh (Methods). Addition of 10 nM and 30 nM NMB increased doublet frequency 1.7-fold (p=0.005; n=7) and 2-fold, respectively (p=0.003; n=7) (FIG. 2e,f). Overall frequency of bursts and doublets together was unchanged (p=0.2; n=7), implying NMB converts inspiratory bursts into sighs; indeed, in some preparations every inspiratory burst was converted to a doublet (FIG. 10). We conclude NMB acts directly on preBötC to increase sighing.

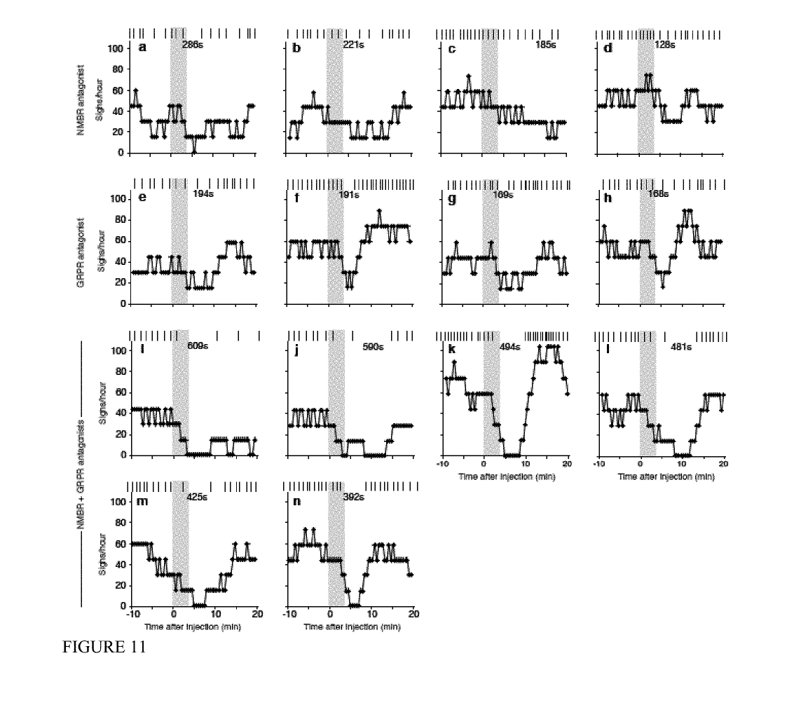

NMBR signaling maintains basal sighing. To determine if NMB signaling is required for sighing, we monitored breathing of awake, unrestrained Nmbr^{-/-} knockout mice. Wild type controls (C57BL/6) sighed 40±11/hour, whereas Nmbr^{-/-} mutants sighed 29±10/hour (n=4; p<0.001) (FIG. 2g). Sighing was also transiently reduced ˜50% in anesthetized rats by NMBR inhibition following bilateral preBötC injection of the antagonist BIM23042 (100 nl, 6 μM) (FIG. 2h, FIG. 11a-d,7a). The antagonist effect was selective for sighing as it did not significantly alter respiratory rate (117±14 vs. 109±6 breaths/minute with antagonist, n=4, p=0.14) or tidal volume (2.1±0.1 vs. 2.0±0.3 ml, n=4, p=0.46), and similar selectivity was observed for the Nmbr mutant (respiratory rate 218±22 vs. 254±22 in Nmbr^{-/-} mice, n=4, p=0.06; tidal volume 0.30±0.03 vs. 0.31±0.04 ml, n=4, p=0.88). Thus NMBR signaling in preBötC maintains basal sighing.

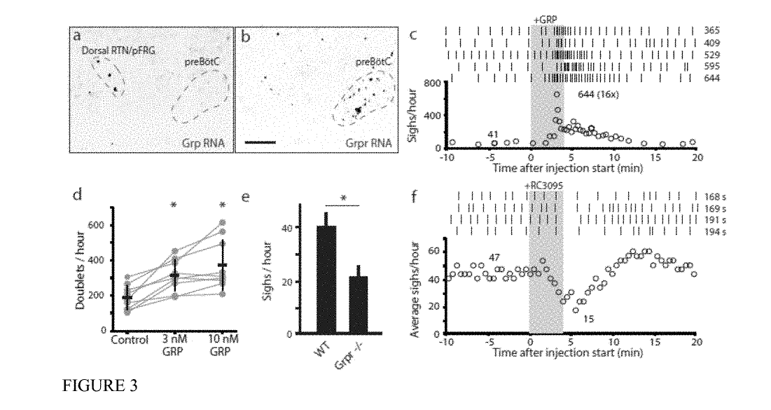

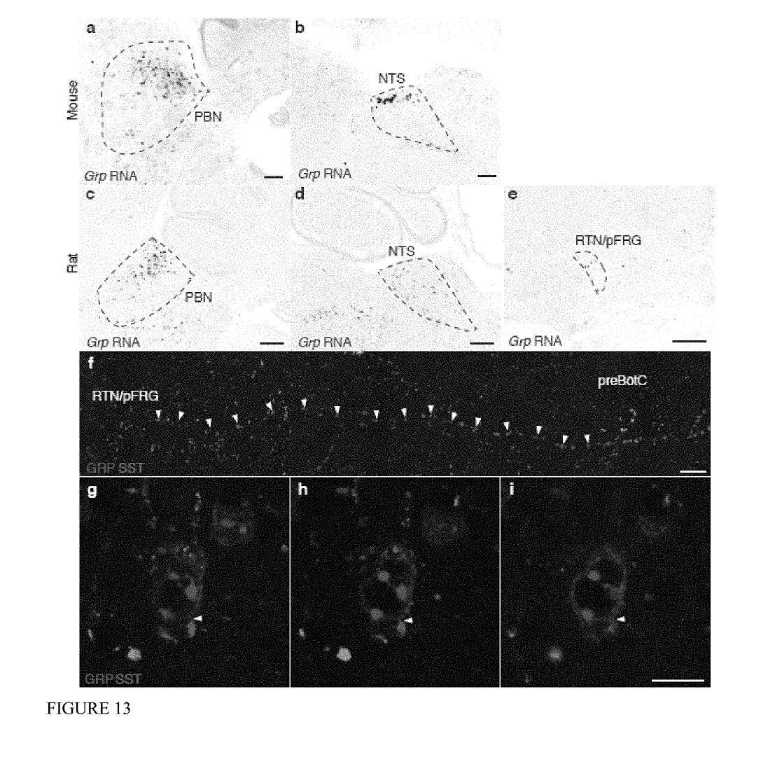

Another bombesin-like neuropeptide pathway also modulates sighing Nmbr mutations and inhibition reduced but did not abolish sighing, suggesting involvement of other pathways. Gastrin-releasing peptide (Grp), the only other bombesin-like neuropeptide gene in mammals, was expressed in several dozen cells in the dorsal RTN/pFRG in mouse (FIG. 3a) and rat (FIG. 13e) plus scattered cells in nucleus tractus solitarius (NTS) and parabrachial nucleus (PBN), two other breathing circuit nuclei (FIG. 13a-d). GRP-positive projections were traced from RTN/pFRG to preBötC, some GRP puncta abutting SST-positive preBötC neurons (FIG. 13f-i). GRP signals through GRPR, the receptor most similar to NMBR. Grpr mRNA was detected in ˜160 mouse preBötC neurons (FIG. 3b and see below), suggesting GRP may also directly modulate preBötC function. To determine if GRP regulates sighing, the neuropeptide (100 nl, 3 μM) was injected bilaterally into preBötC of anesthetized rats. Sighing increased 8-16-fold (n=5; FIG. 3c, FIG. 9f-j). GRP (3 nM) application to mouse preBötC brain slices also increased sighing, showing 1.7-fold more doublets (p=0.003; n=9; FIG. 3d). Thus, GRP can induce sighing through direct modulation of preBötC neurons, like NMB. To determine if GRPR signaling is required for sighing, we monitored breathing in Grpr−/− knockout mice. Their basal sigh rate (22±9/hour, n=4) was half that of control wild type mice (FIG. 3e), whereas eupneic breathing appeared normal (respiratory rate 218±22 vs. 210±16 in Grpr−/−, n=4, p=0.57; tidal volume 0.30±0.03 vs. 0.28±0.01 ml, n=4, p=0.23). GRPR inhibition by bilateral preBötC injection of antagonist RC3095 (100 nl, 6 μM) in anesthetized rats also transiently decreased sighing by ˜50% (n=4), followed by rapid rebound and overshoot (FIG. 3f, FIG. 11e-h). There was no significant change in other respiratory parameters (respiratory rate 117±12 vs. 111±11 with antagonist, n=4, p=0.34; tidal volume 2.0±0.2 vs. 1.9±0.1 ml, n=4, p=0.11). Thus, GRPR signaling in preBötC also maintains basal sighing. Expression patterns, loss-of-function phenotypes and localized pharmacological manipulations of NMBR and GRPR signaling in preBötC suggest that NMB-NMBR and GRP-GRPR pathways can independently modulate sighing.

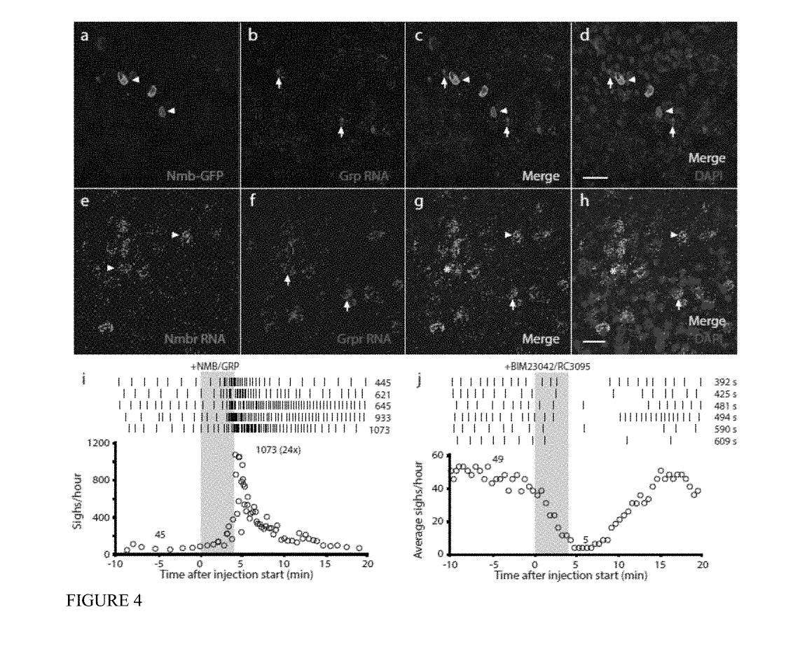

NMBR and GRPR are the critical pathways in sighing To explore the relationship between NMB and GRP pathways, we compared expression patterns of the neuropeptides and receptors within mouse RTN/pFRG and preBötC. Nmb and Grp were detected in non-overlapping neuronal subpopulations, with Nmb neurons distributed throughout RTN/pFRG and Grp neurons restricted to the dorsal domain (FIG. 4a-d). In contrast, receptor expression patterns in preBötC overlapped (FIG. 4e-h), with 40±16 neurons expressing Nmbr, 113±45 expressing Grpr, and 49±9 expressing both (n=3). To explore functional interactions, we injected both neuropeptides into preBötC of anesthetized rats. Sigh rate increased 12-24-fold, similar or slightly beyond that of either neuropeptide alone (FIG. 4i, FIG. 9k-o). When NMBR and GRPR pathways were simultaneously inhibited by bilateral injection of both antagonists, BIM23042 (100 nl, 6 μM) and RC3095 (100 nl, 6 μM), sighing was severely reduced or eliminated (n=6; FIG. 4j, FIG. 11i-n). Thus, NMBR and GRPR pathways can independently modulate sighing, and together are required for basal sighing in vivo.

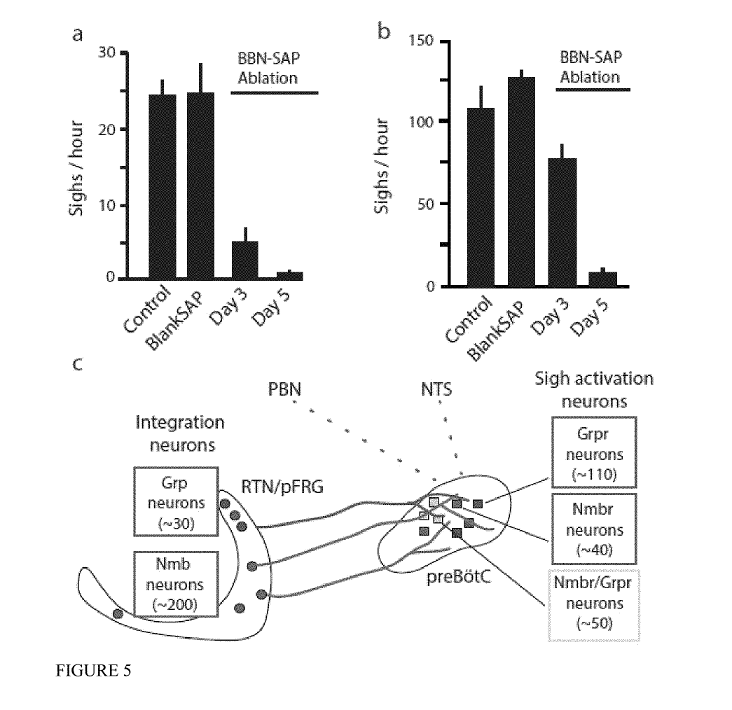

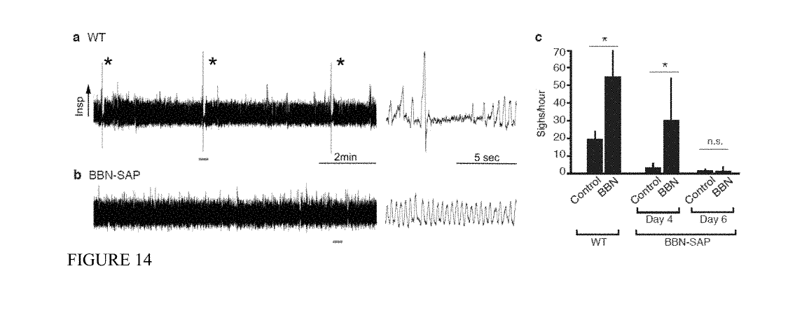

Effect of NMBR and GRPR neuron ablation To determine if preBötC NMBR- and GRPR-expressing neurons function specifically in sigh control, we ablated them using bombesin (BBN), which binds both receptors, conjugated to saporin (BBN-SAP), a ribosomal toxin that induces neuron death when internalized. Three days after bilateral BBN-SAP injection (200 nl, 6.15 ng/side) into preBötC of rats, sighing was reduced ˜80%, from 24±1/hour before injection to 5±2/hour three days after injection (p=5×10⁻⁶; n=7) (FIG. 5a). The effect was selective as other aspects of breathing and behavior appeared normal. Five days after injection, sighing was almost completely (˜95%) abolished, decreasing to 0.6±0.3/hour (p=10⁻⁸; n=6; FIG. 5a). Other aspects of breathing and behavior again appeared generally intact (FIG. 14a, b). However, after five days we noted occasional brief episodes of apneas or disordered breathing, possibly a consequence of the loss of sighing. Ablation prevented sigh induction by exogenous BBN infusion into the cisterna magna, confirming preBötC Nmbr- and Grpr-expressing neurons were eliminated (FIG. 14c). However, BBN infusion still triggered intense scratching and licking, demonstrating that the Grpr and Nmbr neurons outside the preBötC required for these behaviors remained intact. We conclude that preBötC Nmbr and Grpr-expressing neurons have a critical and selective function in basal sighing.

NMBR and GRPR neurons are also critical for hypoxia-induced sighing To determine if the Nmbr and Grpr-expressing neurons are also important for physiologically-induced sighs, we examined BBN-SAP rats exposed to hypoxia (8% O₂). In control rats injected with unconjugated SAP, sighing increased from 24±5 to 140±8/hour under hypoxia (n=3, p=0.01). In contrast, five days after BBN-SAP injection, sigh rate under hypoxia was 5.1±3.2/hour (n=6, p=0.2; FIG. 5b); three of these rats did not sigh in room air (21% O₂) and no sighs were triggered by hypoxia. In BBN-SAP rats, hypoxia increased respiratory rate from 150±1 to 230±3 breaths/min, demonstrating ventilatory response to hypoxia was intact. Thus, Nmbr and Grpr-expressing neurons are also critical for hypoxia-induced sighing, but not other respiratory responses to hypoxia.

Our results show sighing is controlled by two largely parallel bombesin-like neuropeptide pathways, NMB and GRP, which mediate signaling between key medullary breathing control centers. ˜200 Nmb-expressing and -30 Grp-expressing neurons in neighboring domains of RTN/pFRG, a region implicated in integrating respiratory sensory cues and generation of active expiration_{22,27,29}, project to preBötC, the respiratory rhythm generator. 7% (˜200) of preBötC neurons express Nmbr (˜40 neurons), Grpr (˜110), or both receptors (˜50), activation of which increased sighing 6 to 24-fold, whereas sighing was effectively abolished by inhibition or deletion of the receptors, or ablation of the receptor-expressing neurons.

The above neurons, perhaps with Grp-expressing NTS and PBN neurons, may comprise the core of a peptidergic sigh control circuit, with the neuropeptideexpressing neurons integrating inputs from sites monitoring physiological and perhaps emotional state (FIG. 5c). Excitation of these neurons and secretion of either neuropeptide activates the cognate receptor-expressing preBötC neurons, which initiate sighs by altering activity of other preBötC neurons to convert normal breaths to sighs. This might occur by a burst of NMB and/or GRP secretion triggering a second inspiratory signal in the preBötC during or immediately after the first, resulting in a single, double-size breath. Alternatively, NMB and GRP secretion might be more gradually modulated, causing concentration-dependent bursts in activity of receptor-expressing neurons or shift in preBötC properties toward states favoring more frequent doublet bursts.

One aspect of the circuit already apparent is the central role of two partially-overlapping and closely-related neuropeptide pathways. Do NMB- and GRP-expressing neurons receive different inputs and have distinct sensing functions, and do the three sets of receptor-expressing neurons (NMBR, GRPR, NMBR+GRPR) converge on the same preBötC neurons to effect a sigh, or signal to different preBötC neurons producing distinct types of sighs?

A striking aspect of our results is the selectivity of the circuit for sighing. Inhibition of the pathways, and even ablation of receptor-expressing neurons, had little effect on other aspects of breathing, at least in the short term. Identification of the key neuropeptide pathways provides pharmacologic approaches for controlling excessive sighing and inducing sighs in patients that cannot breathe deeply on their own.

### Methods

Animals. All procedures were carried out in accordance with animal care standards in National Institutes of Health (NIH) guidelines, and approved by the University of California, Los Angeles Animal Research Committee, or Stanford Institutional Animal Care and Use Committee. All mouse strains used were in the C57BL/6 genetic background. Nmb-GFP BAC transgenic mice (Tg(Nmb-EGFP)IT50Gsat/Mmucd), carrying EGFP coding sequence inserted upstream of the Nmb start codon in the BAC, were from the Mutant Mouse Regional Resource Centers (catalog number 030425-UCD, https://www.mmrrc.org). Nmbr^{-/-} and Grpr^{-/-} null mutant mice, in which exon 2 of the endogenous gene was replaced by a neomycin-resistance cassette using homologous recombination, have been described. Male Sprague Dawley rats were from Charles River.

In situ hybridization, immunostaining and reporter expression. For in situ hybridization, mouse brains were harvested and fixed overnight in 4% paraformaldehyde in phosphate buffered saline (PBS), cryopreserved in 30% sucrose and embedded in optical cutting temperature compound (OCT). Transverse sections were cut at 16-20 microns and stored at −80° C. until use. Sections were post-fixed in 4% paraformaldehyde before treatment with hydrochloric acid, proteinase K, and then triethanolamine/acetic anhydride. Hybridization was carried out with in vitro transcribed and digoxygenin-labeled riboprobes at 58° C. overnight. Signal was detected using Alkaline Phosphatase-coupled anti-Digoxigenin (DIG) primary antibody (Roche) and nitro blue tetrazolium chloride and bromochloroindolyl phosphate (NBT/BCIP) Reagent Kit (Roche) or using Horse Radish Peroxidase-coupled anti-DIG primary antibody (Roche) and Tyramide Signal Amplification plus Fluorescent Substrate Kit (PerkinElmer). For double fluorescent in situ hybridization, tissue was harvested, embedded in OCT and then sectioned. Sections were fixed in 4% paraformaldehyde, dehydrated and treated with pretreatment reagent (Advanced Cell Diagnostics). Double fluorescent in situ assay was then performed using proprietary RNAscope technology (Advanced Cell Diagnostics) with cyanine 3-labeled Nmbr probes and fluorescein isothiocyanate-labeled Grpr probes. For Nmb-GFP reporter expression analysis, the brains of Nmb-GFP mice were harvested and fixed overnight in 4% paraformaldehyde and then cryopreserved at 4° C. in 30% sucrose overnight. Tissue was embedded in OCT and sectioned at 10-40 microns. Tissue sections were rinsed with PBT (PBS+0.1% Tween), blocked with 3% bovine serum (BSA) in PBT for 1 hr, and incubated with primary antibody overnight at 4° C. Sections were rinsed in PBT and incubated for 1 hour at room temperature with species-specific secondary antibodies.

Primary antibodies were: chicken anti-GFP (Abeam 13970; used at 1:1000 dilution), goat anti-PHOX2B (Santa Cruz, sc-13224; 1:200 dilution), rabbit anti-NMB (Sigma-Aldrich, SAB1301059; 1:100 dilution), rabbit anti-GRP (Immunostar 20073; 1:4000 dilution), and rat anti-SST (Millipore, MAB354; 1:50 dilution). Secondary antibodies included donkey anti-chicken (Jackson Immuno Research; 1:400 dilution), donkey anti-rat (Jackson Immuno Research; 1:400 dilution), donkey anti-rabbit (Jackson Immuno Research; 1:400 dilution) and donkey anti-goat (Invitrogen; 1:500 dilution). For Nmb-GFP expression analysis in samples prepared by CLARITY, Nmb-GFP mice were perfused with PBS and formaldehyde-acrylamide hydrogel, and brain tissue was harvested and incubated in hydrogel monomer solution at 4° C. for 3 days. Tissue was then embedded in polymerized hydrogel by raising the temperature to 37° C. for 3 h. Blocks of 1 mm thickness were cut and washed in 4% sodium dodecyl sulfate (SDS) in sodium borate buffer at 37° C. for 2-3 weeks. Samples were washed with PBST for 2 days and incubated in FocusClear (CelExplorer), and GFP fluorescence was imaged on a Zeiss LSM780 confocal microscope.

Sigh monitoring and analysis. For awake animals, individual animals were placed in a whole body plethysmography chamber (Buxco) at room temperature (22° C.) in 21% O₂ (for normoxia) or 8% O₂ (for hypoxic challenge) balanced with N₂. Sighs were identified in plethysmography traces by the characteristic biphasic ramp, the augmented flow in the second phase of the inspiratory effort and the prolongation of expiratory time following the event. Sighs were also confirmed by visual monitoring of breathing behavior. Given the high amplitude and distinctive waveform of sighs relative to standard eupneic breaths, sighs were unambiguously identified by both visual and computer-assisted scoring; no difference was detected in direct comparisons between methods or observers so visual scoring was used. Female 8 week old mice (Nmbr^{-/-}, Grpr^{-/-}, or C57BL/6 as wild type control) were allowed to acclimate for 10 minutes in the chamber, and then the first fifteen recorded sighs were used to calculate the sigh rate. Similar sigh rates were observed for each animal when assayed on different days. Rats were allowed to acclimate in the chamber for ˜1 hr, and then baseline sigh rate and respiratory frequency were calculated for the next 2 hrs. For hypoxic (8% O₂) challenge, analysis was continued for 30 minutes under the hypoxic condition. For anesthetized rats, the trachea was cannulated and connected to a pneumotachograph (GM Instruments) to record airflow. A flow calibration was performed after every experiment along with a calculation of tidal volume (V_{T}) by digital integration. To monitor diaphragm activity, wire electrodes (Cooner Wre) were implanted into the diaphragm and electromyogram (EMG) signal sampled at 2 kHz (Powerlab 16SP; AD Instruments). Signal was rectified and digitally integrated (time constant of 0.1) to obtain a moving average using LabChart Pro 8 (AD Instruments) and Igor Pro 6 (Data Matrix) software. Sighs were identified in the airflow measurements as above and validated by double peaks in the EMG recordings.

PreBötC injection of NMB and GRP agonists and antagonists. Male Sprague Dawley rats (n=24) weighing 320-470 g were anesthetized with urethane (1.5 g/kg), isoflurane (0.3-0.7 vol %), and ketamine (20 mg/kg/hour) and injected i.p. with atropine (0.3 mg/kg), then placed in a supine position in a stereotaxic instrument (David Kopf Instruments). A tracheostomy tube was placed in the trachea through the larynx, and the basal aspect of the occipital bone was removed to expose the ventral medulla. Injections were placed 750 μm caudal from the most rostral root of the hypoglossal nerve (RRXII), 2 mm lateral to the midline, and 700 μm dorsal to the ventral medullary surface. Small corrections were made to avoid puncturing blood vessels on the medulla. Microinjection was done using a series of pressure pulses (Picospritzer; Parker-Hannifin) applied to the open end of micropipettes, with air pressure set so that each pulse ejected 5 nl and a total volume of 0.1 μl was injected on each side. The concentration of injected neuropeptides and antagonists were: NMB, 3 μM; GRP, 3 μM; NMB and GRP, 3 μM each; BIM23042, 6 μM; RC3095, 6 μM; BIM23042 and RC3095, 6 μM each. To verify the accuracy of the injections, fluorescent polystyrene beads (0.2 μm FluoSpheres (Invitrogen; catalog #F8811, #F8763 or #F8807); 2-5% vol) were added to the injected solutions, and following injection and physiological measurements the location of the fluorescent beads was visualized in wet tissue double-DAB stained for reelin and choline acetyltransferase (ChAT) to identify the preBötC.

Ablation of NMBR and GRPR-expressing preBötC neurons. Bilateral preBötC injections of saporin conjugated with either bombesin (BBNSAP; Advanced Targeting System; 200 nl, 6.2 ng) or a non-targeted peptide (blank SAP; Advanced Targeting System; 200 nl, 6.2 ng) were performed in 300-350 g rats under anesthesia (ketamine (90 mg/kg), and xylazine (10 mg/kg), administered i.p) using standard aseptic procedures. Rats were positioned on a stereotaxic frame with bregma 5 mm below lambda. The occipital bone was exposed and a small window was opened to perform BBN-SAP injections with a 40 μm diameter tip glass pipette inserted into the preBötC. Coordinates were (in mm): 0.9 rostral, 2.0 lateral, and 2.8 ventral to the obex. The electrode was left in place for 5 min after injection to minimize backflow of solution up the electrode track. After injection, a fine polyethylene cannula was implanted and cemented to the occipital bone to deliver BBN into the fourth ventricle. Neck muscles and skin were sutured back at the end of the surgery and rats were allowed to recover with pain medication, food and water ad libitum. Blank-SAP and BBN-SAP treated rats were tested for hypoxia (8% O2 balanced with nitrogen, 30 minute challenge) five days after surgery. Blank-SAP and BBN-SAP treated rats were also tested for response to BBN infusion in the cistern magna six days after surgery. The cannula implanted in the fourth ventricle was connected to a fine polyethylene tubing under isoflurane anesthesia, and after recovery and placement of rats in a plethysmographic chamber 10 μg of BBN diluted in 20 μl sterile saline was delivered followed by a 20 μl saline washout. Sigh rate and respiratory rate were calculated for 30 minutes following infusion and compared to pre-infusion values.

In vitro slice preparation, recording, and analysis. Rhythmic 550-μm-thick transverse medullary slices containing the preBötC and XII nerve from neonatal C57BL/6 mice (P0-5) were prepared as described previously (Kam et al., 2013). The medullary slice was cut in artificial cerebrospinal fluid (ACSF) containing (in mM): 124 NaCl, 3 KCl, 1.5 CaCl₂, 1 MgSO₄, 25 NaHCO₃, 0.5 NaH₂PO₄, and 30 D-glucose, equilibrated with 95% O₂ and 5% CO₂ (4° C., pH=7.4). For recording, extracellular K+ was raised to 9 mM to replace excitatory afferent drive lost in the cutting process. Slices were perfused at 27° C. and 4 ml/min and allowed to equilibrate for 30 minutes. Respiratory activity reflecting suprathreshold action potential (AP) firing from populations of neurons was recorded as XII bursts from either XII nerve roots and as population activity directly from the preBötC using suction electrodes and a MultiClamp 700A or 700B (Molecular Devices, Sunnyvale, Calif., USA), filtered at 2-4 kHz and digitized at 10 kHz. Digitized data were analyzed off-line using custom procedures written for IgorPro (Wavemetrics, Portland, Oreg., USA). Activity was full-wave rectified and digitally integrated with a Paynter filter with a time constant of 20 ms with either custom built electronics or using custom procedures in MATLAB (Mathworks, Natick, Mass., USA). Burst detection and analysis of respiratory-related activity recorded in full-wave rectified XII output or preBötC population recordings were performed using custom software written in IgorPro. Burst parameters were normalized to the mode of the data in the baseline condition. Although there are several proposed definitions of sighs in slice preparations, here we used “doublets” (double-peaked bursts) as the in vitro signature of sighs because, in our preparations, doublets detected both in preBötC neural population activity measurements and cranial nerve XII output recordings shared the increased inspiratory and expiratory duration of sighs. Furthermore, as demonstrated here, doublet rate increased following application of NMB or GRP to preBötC slice preparations (FIGS. 2, 3, FIG. 10), as did sigh rate in vivo following preBötC injection of the same neuropeptides (FIGS. 2, 3). The frequency and waveform of doublets in slice preparations does not closely match those of sighs in intact animals, presumably due to the absence in vitro of important inputs modulating burst shape; indeed, the doublets more closely resemble sighs in vagotomized animals, where they appear as equal amplitude double-peaked breaths. We scored a burst as a doublet if the burst displayed a second peak that reached 20% or more of the amplitude of the first burst, and this second peak occurred after more than twice the time from start to peak or if the burst had a duration longer than eight times the time from start to peak. All doublets were verified by visual inspection to exclude multipeaked bursts and two bursts that were too far apart. Measured doublet intervals were converted to a calculated per hour doublet rate.

Statistics. Data are represented as mean±standard deviation (SD). Statistical significance was uniformly set at a minimum of p<0.05. For comparisons of two groups, the assumption for normal distribution was determined by the Shapiro-Wilk test with the critical W value set at 5% significance level. The t-tests were conducted, with the exception in FIG. 3e, in which a Mann-Whitney U test was used. For statistical comparisons of more than two groups, an ANOVA was first performed. In most cases, a two-way repeated measures ANOVA was used for comparisons of various parameters in different conditions and for making comparisons across different events. If the null hypothesis (equal means) was rejected, post-hoc paired t-tests were then used for pairwise comparisons of interest. Individual p-values are reported, but Holm-Bonferroni analysis for multiple comparisons was conducted to correct for interactions between the multiple groups. Histograms were normalized by the total sample size to generate plots of the relative frequency of each value where the y-value of each bin represents the fraction of the total number of samples for that experiment. Randomization and blinding were not used. No statistical method was used to predetermine sample size.

1. A method of modulating sigh breathing in a mammal through inhibiting or activating one or both of neuromedin B receptor (NMBR) or gastrin releasing peptide receptor (GRPR) present in neurons, the method comprising:
contacting cells of the preBötzinger Complex body in a mammalian subject with an effective dose of a ligand of NMBR or GRPR.

2. The method of claim 1, wherein the ligand is an agonist.

3. The method of claim 2, wherein the agonist is neuromedin B or a variant thereof.

4. The method of claim 2, wherein the agonist is gastrin releasing peptide.

5. The method of claim 2, wherein the effective dose is sufficient to increase frequency of sighs by at least about 25%.

6. The method of claim 1, wherein the ligand is an antagonist.

7. The method of claim 6, wherein the effective dose decreases hyperventilation.

8. A composition for use in the method of claim 1.

9. The composition of claim 8, comprising an effective dose of a ligand of one or both of neuromedin B receptor (NMBR) or gastrin releasing peptide receptor (GRPR) and a pharmaceutically acceptable carrier.

10. The composition of claim 9, wherein the ligand is identified by the method of contacting one or both of NMBR or GRPR with a candidate ligand, and determining the effect of the candidate ligand on receptor activation.

11. The method of claim 10, wherein the receptor is present on the surface of a cell.

12. The method of claim 10, wherein a candidate ligand is further screened for activity regulating ventilation in vivo or in an in vitro brain slice culture.
